(19) United States
(12) Patent Application Publication
(10) Pub. No.: US 2025/0325592 A1
(43) Pub. Date: Oct. 23, 2025

(54) METHOD FOR INDUCED DIFFERENTIATION OF STEM CELLS INTO HEMATOPOIETIC PROGENITOR CELLS

(71) Applicant: XELLSMART BIOMEDICAL (SUZHOU) CO., LTD., Shanghai (CN)

(72) Inventor: Xiang LI, Shanghai (CN)

(21) Appl. No.: 18/724,541

(22) PCT Filed: Dec. 30, 2022
(86) PCT No.: PCT/CN2022/144143

§ 371 Date: Jun. 26, 2024

(30) Foreign Application Priority Data
Dec. 31, 2021 (CN) 2021116757596

(51) Int. Cl. A61K 35/28; C12N 5/0789

(52) U.S. Cl. CPC A61K 35/28; C12N 5/0647; C12N 2500/32; C12N 2500/38; C12N 2501/125; C12N 2501/155; C12N 2501/165; C12N 2506/02; C12N 2506/45

(57) ABSTRACT

The present disclosure relates to a method for induced differentiation of stem cells into hematopoietic progenitor cells. The method comprises the following steps: culturing pluripotent stem cells to obtain an embryoid body; performing mesoderm differentiation culture on the embryoid body to obtain mesoderm cells; performing hemogenic endothelium differentiation culture on the mesoderm cells to obtain hemogenic endothelial cells; and performing hematopoietic progenitor cell differentiation culture on the hemogenic endothelial cells to obtain hematopoietic progenitor cells. According to the present disclosure, the hematopoietic progenitor cells can be rapidly and efficiently prepared, and the prepared hematopoietic progenitor cells have an ability to stably differentiate into a plurality of different blood cells (including simultaneously having erythroid, myeloid, and lymphoid cells); by optimizing a culture system, the differentiation efficiency is significantly improved and the number of hematopoietic progenitor cells obtained is significantly increased.

### CROSS-REFERENCE TO RELATED APPLICATIONS

The present application is a National Stage Application claiming the priority of co-pending PCT Application No. PCT/CN2022/144143 filed Dec. 30, 2022, which claims priority from Chinese Patent Application No. 202111675759.6, filed Dec. 31, 2021. The priority applications are herein specifically incorporated by reference in their entirety.

### INCORPORATION BY REFERENCE

The instant application contains a Sequence Listing encoded in eXtensible Markup Language (a “xml” file) that is submitted herewith named P24GZ1NW00092US_Sequence_Listing.xml created on Dec. 4, 2024, and 41,478 bytes in size. This sequence listing is incorporated by reference herein.

### TECHNICAL FIELD

The present disclosure relates to the field of cell technology, and particularly, to a method for induced differentiation of stem cells into hematopoietic progenitor cells.

### BACKGROUND

Hematological disorders are diseases that originate in the hematopoietic system or affect the hematopoietic system with abnormal hematologic changes, with common symptoms such as anemia, bleeding, fever, etc. The morbidity of malignancies in children in China is on the rise, and the data as of 2014 show that leukemia leads the malignancies in children in morbidity and accounts for about one third. The efficacy of chemotherapies in clinic against hematological malignancies is usually unsatisfying. Since the first hematopoietic stem cell (HSC) transplantation by Prof. Thomas in mid 1900s, HSC transplantation has been widely used for the clinical treatment of leukemia, and has become one of the effective means for treating diseases such as acute leukemia, malignant lymphoma, and severe aplastic anemia.

Currently, HSCs are mainly derived from cord blood, bone marrow, and peripheral blood. HSC transplantation is mainly classified into autologous and allogeneic HSC transplantations. Although autologous transplantation features the advantageous absence of graft rejection, graft-versus-host disease, and other complications, the shortage of autologous HSCs in cord blood banks greatly limits their clinical applications. Although allogeneic transplantation excels autologous transplantation in long-term efficacy and recurrence, it possesses extremely low match efficiency and limited sources, thus restricting the clinical application of allogeneic HSC transplantation.

Therefore, there is an urgent need in the art for a safe, cost-efficient, and stable source of hematopoietic stem/progenitor cells. Pluripotent stem cells, including embryonic stem cells and induced pluripotent stem cells, can differentiate into various tissues in the body and can thus be used for preparing disease models, assisting drug toxicity studies, and promoting wound repair and treating diseases by replacing damaged or diseased cells via cell transplantation. Hematopoietic stem cells are present in the body for a lifetime. They can differentiate into various cells of the blood system, including erythrocytes, granulocytes, macrophages, monocytes, microglial cells, dendritic cells, B-lymphocytes, T-lymphocytes, NK-lymphocytes, etc., and have great prospects in terms of clinical treatment of hematological disorders, cancers and the like.

Hematopoietic stem cells can rebuild the hematopoietic system, differentiate into hematopoietic cells of various lineages, and maintain their potency. However, the hematopoietic stem cells separated in vitro at a single-cell level can hardly be expanded to a large quantity, whereas the induced differentiation of stem cells in vitro can hardly provide hematopoietic stem cells for long-term rebuilding in vivo are difficult to acquire through but only hematopoietic progenitor cells with the capability of short-term rebuilding in vivo and some properties of hematopoietic stem cells. Hematopoietic progenitor cells can differentiate into blood cells of various lineages and can be used for hematological disorders.

Currently, the main approaches for inducing the differentiation of human pluripotent stem cells into hematopoietic progenitor cells include the embryoid body differentiation method and the stromal cell co-incubation method. The methods also have some drawbacks: The embryoid body method usually consumes a large quantity of pluripotent stem cells, which lead to inconsistency of the differentiation stages and thus low differentiation efficiency and excessive time consumption; the stromal cell co-incubation method possesses an unstable efficiency and may introduce animal-derived components, serum-containing culture systems, or trophoblast cells, and are therefore unsuitable for subsequent production of clinical-grade cell formulations. Therefore, there is an urgent need in the art for a highly efficient method for preparing hematopoietic progenitor cells that are chemically defined and can rapidly and stably differentiate in serum-free conditions.

### SUMMARY

The present disclosure is intended to provide a highly efficient method for preparing hematopoietic progenitor cells that are chemically defined and can rapidly and stably differentiate in serum-free conditions.

For this purpose, the present disclosure provides the following embodiments:

In first aspect, the present disclosure provides a method for preparing a hematopoietic progenitor cell, comprising:
(1) culturing a pluripotent stem cell to give an embryoid body;
(2) subjecting the embryoid body to mesoderm differentiation culture to give a mesodermal cell;
(3) subjecting the mesodermal cell to hemogenic endothelium differentiation culture to give a hemogenic endothelial cell; and
(4) subjecting the hemogenic endothelial cell to hematopoietic progenitor cell differentiation culture to give a hematopoietic progenitor cell.

Preferably, the culture system in step (1) comprises a ROCK inhibitor. Preferably, the ROCK inhibitor includes, but is not limited to, at least one selected from the group consisting of: blebbistatin, HA-100, Y-27632, HA-1077, KD-025, Y-33075, and narciclasine. Preferably, the concentration of the ROCK inhibitor is 1-50 μM, more preferably 5-20 μM, and even more preferably 10 μM. Preferably, the ROCK inhibitor is Y-27632 at a concentration of 1-50 μM, more preferably 5-20 μM, and even more preferably 10 μM.

Preferably, the culture system in step (1) is a pluripotent stem cell culture medium comprising the ROCK inhibitor. Preferably, the pluripotent stem cell culture medium includes, but is not limited to: E8 medium, mTESR medium, StemFit Basic 03, StemFit Basic 04, NutriStem hPSC XF medium, StemMACS iPS-Brew medium, Stem-Partner ACF medium, TeSR-AOF medium, and TeSR2 medium.

Preferably, the culture time in step (1) is 12-30 hours, more preferably 15-24 hours, and even more preferably 20-24 hours.

Preferably, the culture system in step (2) comprises BMP4 and/or a GSK-3β inhibitor.

Preferably, the culture system in step (2) comprises a GSK-3β inhibitor.

Preferably, the concentration of BMP4 is 0-100 ng/mL; more preferably, the concentration of BMP4 is 5-50 ng/mL; even more preferably, the concentration of BMP4 is 10-20 ng/mL.

Preferably, the GSK-3β inhibitor includes, but is not limited to, at least one selected from the group consisting of: B216763, TWS119, NP031112, SB216763, CHIR-98014, AZD2858, AZD1080, SB415286, LY2090314, and CHIR-99021. Preferably, the concentration of the GSK-3β inhibitor is 0.5-20 μM, more preferably 1-10 μM, and even more preferably 3-5 μM. Preferably, the GSK-3β inhibitor is CHIR-99021 at a concentration of 0.5-20 μM, more preferably 1-10 μM, and even more preferably 3-5 μM.

Preferably, the culture system in step (2) is free of antibiotics. The antibiotics include, but are not limited to: amphotericin, nystatin, gentamicin, tetracycline, erythromycin, penicillin, and streptomycin. Preferably, the culture system in step (2) is free of penicillin and streptomycin.

Preferably, the antibiotic is penicillin and/or streptomycin. More preferably, the antibiotic is penicillin-streptomycin.

Preferably, the culture system in step (2) is free of monothioglycerol (MTG). Preferably, the culture system in step (2) is a basal medium comprising BMP4 and/or GSK-3β inhibitor.

Preferably, the culture system in step (2) or the basal medium comprises at least one, at least two, at least three, or four selected from the group consisting of: B27 additive, a non-essential amino acid, glutamine, and vitamin C. Preferably, the B27 additive is a B27 additive free of vitamin A. Preferably, the concentration of the added B27 additive (e.g., B27 additive free of vitamin A) is 0.5-10%, more preferably 1-5%, and even more preferably 2%. The concentration of the added non-essential amino acid is 0.2-10%, more preferably 0.5-2%, and even more preferably 1%. The concentration of the added glutamine is 0.2-10%, more preferably 0.5-2%, and even more preferably 1%. The above percentages are mass-to-volume ratios, and the concentration of the added vitamin C is 10-100 μg/mL, more preferably 20-50 μg/mL, and even more preferably 50 μg/mL.

Preferably, the culture time in step (2) is 18-54 hours, more preferably 20-48 hours, and even more preferably 24-48 hours.

Preferably, the culture system in step (3) comprises at least one, at least two, at least three, or four selected from the group consisting of: BMP4, vascular endothelial growth factor, fibroblast growth factor, and a TGFβ/ALK inhibitor.

Preferably, the concentration of BMP4 is 1-50 ng/mL; more preferably, the concentration is 2-20 ng/mL; even more preferably, the concentration is 5-10 ng/mL.

Preferably, vascular endothelial growth factor (VEGF) includes, but is not limited to, at least one selected from the group consisting of: VEGF-A, VEGF-165, VEGF-183, VEGF-110, VEGF-121, VEGF-B, VEGF-C, VEGF-D, VEGF-E, and placental growth factor. Preferably, the concentration of VEGF is 5-100 ng/mL; more preferably, the concentration is 10-50 ng/mL; even more preferably, the concentration is 20-50 ng/mL. Preferably, VEGF is VEGF-165 or VEGF-A, and the concentration is 5-100 ng/mL; more preferably, the concentration is 10-50 ng/mL; even more preferably, the concentration is 20-50 ng/mL.

Preferably, fibroblast growth factor (FGF) is a polypeptide consisting of about 150-200 amino acids present in two closely related forms, basic fibroblast growth factor (bFGF) and acidic fibroblast growth factor (aFGF), and the concentration of FGF (acidic fibroblast growth factor and/or basic fibroblast growth factor) is 5-100 ng/mL; more preferably, the concentration is 10-50 ng/mL; even more preferably, the concentration is 20-50 ng/mL. Preferably, FGF is FGF-2 (bFGF) at a concentration of 5-100 ng/mL; more preferably, the concentration is 10-50 ng/mL; even more preferably, the concentration is 20-50 ng/mL.

Preferably, the TGFβ/ALK inhibitor include, but are not limited to, at least one selected from the group consisting of: SB431542, SB-505, A-83-01, GW6604, IN-1130, Ki26894, LY2157299, LY364947 (HTS-466284), LY550410, LY573636, LY580276, NPC-30345, SB-505124, SD-093, Sm16, SM305, SX-007, Antp-Sm2A, and LY2109761. Preferably, the concentration of the TGFβ/ALK inhibitor is 1-50 μM, more preferably 5-20 μM, and even more preferably 5-10 μM. Preferably, the TGFβ/ALK inhibitor is SB431542 at a concentration of 1-50 μM, more preferably 5-20 μM, and even more preferably 5-10 μM.

Preferably, the culture system in step (3) is free of antibiotics. The antibiotics include, but are not limited to: amphotericin, nystatin, gentamicin, tetracycline, erythromycin, penicillin, and streptomycin. Preferably, the culture system in step (3) is free of penicillin and streptomycin.

Preferably, the antibiotic is penicillin and/or streptomycin. More preferably, the antibiotic is penicillin-streptomycin.

Preferably, the culture system in step (3) is free of monothioglycerol (MTG).

Preferably, the culture system in step (3) is a basal medium comprising at least one, at least two, at least three, or four selected from the group consisting of: BMP4, vascular endothelial growth factor, fibroblast growth factor, and a TGFβ/ALK inhibitor.

Preferably, the culture system in step (3) or the basal medium comprises at least one, at least two, three, or four selected from the group consisting of: B27 additive, a non-essential amino acid, glutamine, and vitamin C. Preferably, the B27 additive is a B27 additive free of vitamin A.

Preferably, the concentration of the added B27 additive (e.g., B27 additive free of vitamin A) is 0.5-10%, more preferably 1-5%, and even more preferably 2%. The concentration of the added non-essential amino acid is 0.2-10%, more preferably 0.5-2%, and even more preferably 1%. The concentration of the added glutamine is 0.2-10%, more preferably 0.5-2%, and even more preferably 1%. The above percentages are mass-to-volume ratios, and the concentration of the added vitamin C is 10-100 μg/mL, more preferably 20-50 μg/mL, and even more preferably 50 μg/mL.

Preferably, the culture time in step (3) is 2-6 days, more preferably 3-5 days, and even more preferably 4 days.

Preferably, the culture system in step (4) comprises at least one, at least two, or three selected from the group consisting of: BMP4, vascular endothelial growth factor, and stem cell factor.

Preferably, the concentration of BMP4 is 1-50 ng/mL; more preferably, the concentration is 2-20 ng/mL; even more preferably, the concentration is 5-10 ng/mL.

Preferably, vascular endothelial growth factor (VEGF) includes, but is not limited to, at least one selected from the group consisting of: VEGF-A, VEGF-165, VEGF-183, VEGF-110, VEGF-121, VEGF-B, VEGF-C, VEGF-D, VEGF-E, and placental growth factor. Preferably, the concentration of VEGF is 1-50 ng/mL; more preferably, the concentration is 5-20 ng/mL; even more preferably, the concentration is 10 ng/mL. Preferably, VEGF is VEGF-165 or VEGF-A, and the concentration is 1-50 ng/mL; more preferably, the concentration is 5-20 ng/mL; even more preferably, the concentration is 10 ng/mL.

Preferably, the concentration of stem cell factor (SCF) is 5-100 ng/mL; more preferably, the concentration is 10-50 ng/mL; even more preferably, the concentration is 20-50 ng/mL.

Preferably, the culture system in step (4) is free of antibiotics. The antibiotics include, but are not limited to: amphotericin, nystatin, gentamicin, tetracycline, erythromycin, penicillin, and streptomycin. Preferably, the culture system in step (4) is free of penicillin and streptomycin.

Preferably, the antibiotic is penicillin and/or streptomycin. More preferably, the antibiotic is penicillin-streptomycin.

Preferably, the culture system in step (4) is free of monothioglycerol (MTG).

Preferably, the culture system in step (4) is a basal medium comprising at least one, at least two, or three selected from the group consisting of: BMP4, vascular endothelial growth factor, and stem cell factor.

Preferably, the culture system or the basal medium in step (4) comprises at least one, at least two, at least three, at least four, at least five, at least six, or seven selected from the group consisting of: B27 additive, non-essential amino acids, glutamine, vitamin C, N-acetyl-L-cysteine (NAC), minocycline hydrochloride, and insulin-transferrin-selenium (ITS-G). Preferably, the B27 additive is a B27 additive free of vitamin A. Preferably, the concentration of the added B27 additive (e.g., B27 additive free of vitamin A) is 0.5-10%, more preferably 1-5%, and even more preferably 2%. The concentration of the added non-essential amino acid is 0.2-10%, more preferably 0.5-2%, and even more preferably 1%. The concentration of the added glutamine is 0.2-10%, more preferably 0.5-2%, and even more preferably 1%. The above percentages are mass-to-volume ratios, and the concentration of the added vitamin C is 10-100 μg/mL, more preferably 20-50 μg/mL, and even more preferably 50 μg/mL. The concentration of the added N-acetyl-L-cysteine is 5-100 μM, more preferably 10-50 μM, and even more preferably 30 μM. The concentration of the added minocycline hydrochloride is 0.1-20 μM, more preferably 1-5 μM, and even more preferably 2 μM. The volume percentage of the added insulin-transferrin-selenium is 0.2-10%, more preferably 0.5-2%, and still more preferably 1%.

Preferably, the culture time in step (4) is 4-8 days, more preferably 5-7 days, and even more preferably 6 days.

Preferably, the method for preparing a hematopoietic progenitor cell is a method for preparing a hematopoietic progenitor cell in a serum-free condition.

Preferably, the method for preparing a hematopoietic progenitor cell is a method for preparing a hematopoietic progenitor cell in the absence of vitamin A.

Preferably, the method for preparing a hematopoietic progenitor cell is a method for preparing a hematopoietic progenitor cell in the absence of antibiotics.

Preferably, the method for preparing a hematopoietic progenitor cell is a method for preparing a hematopoietic progenitor cell in the absence of monothioglycerol.

Preferably, the method for preparing a hematopoietic progenitor cell requires no purification and/or enrichment procedures.

Preferably, the method for preparing a hematopoietic progenitor cell is a method for preparing a hematopoietic progenitor cell in a trophoblast-free condition.

Preferably, the culture in any one of steps (1) to (4) is a suspension culture or an adherent culture.

In second aspect, the present disclosure provides a kit for preparing a hematopoietic progenitor cell, comprising at least one, at least two, at least three, or four of an embryoid body culture system, a mesoderm differentiation culture system, a hemogenic endothelium differentiation culture system, and a hematopoietic progenitor cell differentiation culture system. Preferably, the kit comprises a mesoderm differentiation culture system and a hemogenic endothelium differentiation culture system. Preferably, the kit further comprises a hematopoietic progenitor cell differentiation culture system. Preferably, the kit further comprises an embryoid body culture system.

Preferably, the kit is used in the method for preparing a hematopoietic progenitor cell according to the first aspect of the present disclosure.

Preferably, the embryoid body culture system comprises ROCK inhibitor. Preferably, the ROCK inhibitor includes, but is not limited to, at least one selected from the group consisting of: blebbistatin, HA-100, Y-27632, HA-1077, KD-025, Y-33075, and narciclasine. Preferably, the concentration of the ROCK inhibitor is 1-50 μM, more preferably 5-20 μM, and even more preferably 10 μM. Preferably, the ROCK inhibitor is Y-27632 at a concentration of 1-50 μM, more preferably 5-20 μM, and even more preferably 10 μM.

Preferably, the embryoid body culture system is a pluripotent stem cell culture medium comprising the ROCK inhibitor. Preferably, the pluripotent stem cell culture medium includes, but is not limited to: E8 medium, mTESR medium, StemFit Basic 03, StemFit Basic 04, NutriStem hPSC XF medium, StemMACS iPS-Brew medium, Stem-Partner ACF medium, TeSR-AOF medium, and TeSR2 medium.

Preferably, the mesoderm differentiation culture system comprises BMP4 and/or a GSK-3β inhibitor. Preferably, the mesoderm differentiation culture system comprises a GSK-3β inhibitor Preferably, the concentration of BMP4 is 0-100 ng/mL; more preferably, the concentration of BMP4 is 5-50 ng/mL; even more preferably, the concentration of BMP4 is 10-20 ng/mL.

Preferably, the GSK-3β inhibitor includes, but is not limited to, at least one selected from the group consisting of: B216763, TWS119, NP031112, SB216763, CHIR-98014, AZD2858, AZD1080, SB415286, LY2090314, and CHIR-99021. Preferably, the concentration of the GSK-3β inhibitor is 0.5-20 μM, more preferably 1-10 μM, and even more preferably 3-5 μM. The GSK-3β inhibitor is CHIR-99021 at a concentration of 0.5-20 μM, more preferably 1-10 μM, and even more preferably 3-5 μM.

Preferably, the mesoderm differentiation culture system is free of antibiotics. The antibiotics include, but are not limited to: amphotericin, nystatin, gentamicin, tetracycline, erythromycin, penicillin, and streptomycin. Preferably, the mesoderm differentiation culture system is free of penicillin and streptomycin. Preferably, the antibiotic is penicillin and/or streptomycin. More preferably, the antibiotic is penicillin-streptomycin.

Preferably, the mesoderm differentiation culture system is free of monothioglycerol (MTG).

Preferably, the mesoderm differentiation culture system is a basal medium comprising BMP4 and/or a GSK-3β inhibitor.

Preferably, the mesoderm differentiation culture system comprises at least one, at least two, at least three, or four selected from the group consisting of: B27 additive, a non-essential amino acid, glutamine, and vitamin C. Preferably, the B27 additive is a B27 additive free of vitamin A. Preferably, the concentration of the added B27 additive (e.g., B27 additive free of vitamin A) is 0.5-10%, more preferably 1-5%, and even more preferably 2%. The concentration of the added non-essential amino acid is 0.2-10%, more preferably 0.5-2%, and even more preferably 1%. The concentration of the added glutamine is 0.2-10%, more preferably 0.5-2%, and even more preferably 1%. The above percentages are mass-to-volume ratios, and the concentration of the added vitamin C is 10-100 μg/mL, more preferably 20-50 μg/mL, and even more preferably 50 μg/mL.

Preferably, the hemogenic endothelium differentiation culture system comprises at least one, at least two, at least three, or four selected from the group consisting of: BMP4, vascular endothelial growth factor, fibroblast growth factor, and a TGFβ/ALK inhibitor.

Preferably, the concentration of BMP4 is 1-50 ng/mL; more preferably, the concentration is 2-20 ng/mL; even more preferably, the concentration is 5-10 ng/mL.

Preferably, vascular endothelial growth factor (VEGF) includes, but is not limited to, at least one selected from the group consisting of: VEGF-A, VEGF-165, VEGF-183, VEGF-110, VEGF-121, VEGF-B, VEGF-C, VEGF-D, VEGF-E, and placental growth factor. Preferably, the concentration of VEGF is 5-100 ng/mL; more preferably, the concentration is 10-50 ng/mL; even more preferably, the concentration is 20-50 ng/mL. Preferably, VEGF is VEGF-165 or VEGF-A, and the concentration is 5-100 ng/mL; more preferably, the concentration is 10-50 ng/mL; even more preferably, the concentration is 20-50 ng/mL.

Preferably, fibroblast growth factor (FGF) is a polypeptide consisting of about 150-200 amino acids present in two closely related forms, basic fibroblast growth factor (bFGF) and acidic fibroblast growth factor (aFGF), and the concentration of FGF (acidic fibroblast growth factor and/or basic fibroblast growth factor) is 5-100 ng/mL; more preferably, the concentration is 10-50 ng/mL; even more preferably, the concentration is 20-50 ng/mL. Preferably, FGF is FGF-2 (bFGF) at a concentration of 5-100 ng/mL; more preferably, the concentration is 10-50 ng/mL; even more preferably, the concentration is 20-50 ng/mL.

Preferably, the TGFβ/ALK inhibitor include, but are not limited to, at least one selected from the group consisting of: SB431542, SB-505, A-83-01, GW6604, IN-1130, Ki26894, LY2157299, LY364947 (HTS-466284), LY550410, LY573636, LY580276, NPC-30345, SB-505124, SD-093, Sm16, SM305, SX-007, Antp-Sm2A, and LY2109761. Preferably, the concentration of the TGFβ/ALK inhibitor is 1-50 μM, more preferably 5-20 μM, and even more preferably 5-10 μM. Preferably, the TGFβ/ALK inhibitor is SB431542 at a concentration of 1-50 μM, more preferably 5-20 μM, and even more preferably 5-10 μM.

Preferably, the hemogenic endothelium differentiation culture system is free of antibiotics. The antibiotics include, but are not limited to: amphotericin, nystatin, gentamicin, tetracycline, erythromycin, penicillin, and streptomycin. Preferably, the hemogenic endothelium differentiation culture system is free of penicillin and streptomycin. Preferably, the antibiotic is penicillin and/or streptomycin. More preferably, the antibiotic is penicillin-streptomycin.

Preferably, the hemogenic endothelium differentiation culture system is free of monothioglycerol (MTG).

Preferably, the hemogenic endothelium differentiation culture system is a basal medium comprising at least one, at least two, at least three, or four selected from the group consisting of: BMP4, vascular endothelial growth factor, fibroblast growth factor, and a TGFβ/ALK inhibitor. Preferably, the hemogenic endothelium differentiation culture system comprises at least one, at least two, three, or four selected from the group consisting of: B27 additive, a non-essential amino acid, glutamine, and vitamin C. Preferably, the B27 additive is a B27 additive free of vitamin A. Preferably, the concentration of the added B27 additive (e.g., B27 additive free of vitamin A) is 0.5-10%, more preferably 1-5%, and even more preferably 2%. The concentration of the added non-essential amino acid is 0.2-10%, more preferably 0.5-2%, and even more preferably 1%. The concentration of the added glutamine is 0.2-10%, more preferably 0.5-2%, and even more preferably 1%. The above percentages are mass-to-volume ratios, and the concentration of the added vitamin C is 10-100 μg/mL, more preferably 20-50 μg/mL, and even more preferably 50 μg/mL.

Preferably, the hematopoietic progenitor cell differentiation culture system comprises at least one, at least two, or three selected from the group consisting of: BMP4, vascular endothelial growth factor, and stem cell factor.

Preferably, the concentration of BMP4 is 1-50 ng/mL; more preferably, the concentration is 2-20 ng/mL; even more preferably, the concentration is 5-10 ng/mL.

Preferably, vascular endothelial growth factor (VEGF) includes, but is not limited to, at least one selected from the group consisting of: VEGF-A, VEGF-165, VEGF-183, VEGF-110, VEGF-121, VEGF-B, VEGF-C, VEGF-D, VEGF-E, and placental growth factor. Preferably, the concentration of VEGF is 1-50 ng/mL; more preferably, the concentration is 5-20 ng/mL; even more preferably, the concentration is 10 ng/mL. Preferably, VEGF is VEGF-165 or VEGF-A, and the concentration is 1-50 ng/mL; more preferably, the concentration is 5-20 ng/mL; even more preferably, the concentration is 10 ng/mL.

Preferably, the concentration of stem cell factor (SCF) is 5-100 ng/mL; more preferably, the concentration is 10-50 ng/mL; even more preferably, the concentration is 20-50 ng/mL. Preferably, the hematopoietic progenitor cell differentiation culture system is free of antibiotics. The antibiotics include, but are not limited to: amphotericin, nystatin, gentamicin, tetracycline, erythromycin, penicillin, and streptomycin. Preferably, the hematopoietic progenitor cell differentiation culture system is free of penicillin and streptomycin. Preferably, the antibiotic is penicillin and/or streptomycin. More preferably, the antibiotic is penicillin-streptomycin. Preferably, the hematopoietic progenitor cell differentiation culture system is free of monothioglycerol (MTG).

Preferably, the hematopoietic progenitor cell differentiation culture system is a basal medium comprising at least one, at least two, or three selected from the group consisting of: BMP4, vascular endothelial growth factor, and stem cell factor.

Preferably, the hematopoietic progenitor cell differentiation culture system comprises at least one, at least two, at least three, at least four, at least five, at least six, or seven selected from the group consisting of: B27 additive, non-essential amino acids, glutamine, vitamin C, N-acetyl-L-cysteine (NAC), minocycline hydrochloride, and insulin-transferrin-selenium. Preferably, the B27 additive is a B27 additive free of vitamin A. Preferably, the concentration of the added B27 additive (e.g., B27 additive free of vitamin A) is 0.5-10%, more preferably 1-5%, and even more preferably 2%. The concentration of the added non-essential amino acid is 0.2-10%, more preferably 0.5-2%, and even more preferably 1%. The concentration of the added glutamine is 0.2-10%, more preferably 0.5-2%, and even more preferably 1%. The above percentages are mass-to-volume ratios, and the concentration of the added vitamin C is 10-100 μg/mL, more preferably 20-50 μg/mL, and even more preferably 50 μg/mL. The concentration of the added N-acetyl-L-cysteine is 5-100 μM, more preferably 10-50 μM, and even more preferably 30 μM. The concentration of the added minocycline hydrochloride is 0.1-20 μM, more preferably 1-5 μM, and even more preferably 2 μM. The volume percentage of the added insulin-transferrin-selenium is 0.2-10%, more preferably 0.5-2%, and still more preferably 1%.

In third aspect, the present disclosure provides a hematopoietic progenitor cell prepared by the method according to the first aspect of the present disclosure or using the kit according to the second aspect of the present disclosure.

Preferably, the hematopoietic progenitor cell has the CD34+ phenotype.

Preferably, the hematopoietic progenitor cell has the CD43+ phenotype.

Preferably, the hematopoietic progenitor cell has the CD45+ phenotype.

Preferably, the hematopoietic progenitor cell has the CD34+/CD45+ phenotype.

Preferably, the hematopoietic progenitor cell has the CD34+/CD43+ phenotype.

Preferably, the hematopoietic progenitor cell has the CD43+/CD45+ phenotype.

Preferably, the hematopoietic progenitor cell has the CD34+/CD117+ phenotype.

Preferably, the hematopoietic progenitor cell has the CD45+/CD117+ phenotype.

Preferably, the hematopoietic progenitor cell has the CD43+/CD117+ phenotype.

Preferably, the hematopoietic progenitor cell is a human hematopoietic progenitor cell.

Preferably, the hematopoietic progenitor cell is a hematopoietic progenitor cell population.

Preferably, the hematopoietic progenitor cell has 1, 2, 3, 4, 5, 6, 7, 8, or 9 features selected from the following group (A) consisting of:
(i) 80% or greater, 85% or greater, 90% or greater, 91% or greater, 92% or greater, 93% or greater, 94% or greater, 95% or greater, 96% or greater, 97% or greater, 98% or greater, or 99% or greater of the cells have the hematopoietic progenitor cell surface antigen CD34+;
(ii) 80% or greater, 85% or greater, 90% or greater, 91% or greater, 92% or greater, 93% or greater, 94% or greater, 95% or greater, 96% or greater, 97% or greater, 98% or greater, or 99% or greater of the cells have the hematopoietic progenitor cell surface antigen CD43+;
(iii) 80% or greater, 85% or greater, 90% or greater, 91% or greater, 92% or greater, 93% or greater, 94% or greater, 95% or greater, 96% or greater, 97% or greater, 98% or greater, or 99% or greater of the cells have the hematopoietic progenitor cell surface antigen CD45+;
(iv) 80% or greater, 85% or greater, 90% or greater, 91% or greater, 92% or greater, 93% or greater, 94% or greater, 95% or greater, 96% or greater, 97% or greater, 98% or greater, or 99% or greater of the cells have the hematopoietic progenitor cell surface antigen combination CD34+/CD45+;
(v) 80% or greater, 85% or greater, 90% or greater, 91% or greater, 92% or greater, 93% or greater, 94% or greater, 95% or greater, 96% or greater, 97% or greater, 98% or greater, or 99% or greater of the cells have the hematopoietic progenitor cell surface antigen combination CD34+/CD43+;
(vi) 80% or greater, 85% or greater, 90% or greater, 91% or greater, 92% or greater, 93% or greater, 94% or greater, 95% or greater, 96% or greater, 97% or greater, 98% or greater, or 99% or greater of the cells have the hematopoietic progenitor cell surface antigen combination CD43+/CD45+;
(vii) 30% or greater, 35% or greater, 40% or greater, 45% or greater, 50% or greater, or 55% or greater of the cells have the hematopoietic progenitor cell surface antigen combination CD45+/CD117+; (viii) 30% or greater, 35% or greater, 40% or greater, 45% or greater, 50% or greater, or 55% or greater of the cells have the hematopoietic progenitor cell surface antigen combination CD34+/CD117+; and
(ix) 30% or greater, 35% or greater, 40% or greater, 45% or greater, 50% or greater, or 55% or greater of the cells have the hematopoietic progenitor cell surface antigen combination CD43+/CD117+.

Preferably, the method according to the first aspect of the present disclosure can give a hematopoietic progenitor cell having 1, 2, 3, 4, 5, 6, 7, 8, or 9 of the above features (i)-(ix) without purification and/or enrichment procedures.

Preferably, the hematopoietic progenitor cell has the ability to differentiate into a CD34+/CD45+ blood precursor cell.

Preferably, the hematopoietic progenitor cell has the ability to differentiate into an erythroid blood cell.

Preferably, the hematopoietic progenitor cell has the ability to differentiate into a myeloid blood cell.

Preferably, the hematopoietic progenitor cell also has the ability to differentiate into a lymphocyte.

In fourth aspect, the present disclosure provides a product comprising the hematopoietic progenitor cell according to the third aspect of the present disclosure.

Preferably, the product is a pharmaceutical composition comprising: a hematopoietic progenitor cell according to the third aspect of the present disclosure, in combination with a pharmaceutically acceptable carrier.

Preferably, the pharmaceutical composition is a liquid formulation. Preferably, the pharmaceutical composition is a cell-based formulation. Preferably, the pharmaceutical composition is an intravenous injection. Preferably, the pharmaceutically acceptable carrier includes, but is not limited to: saline, buffer, dextrose, water, DMSO, and a combination thereof. Preferably, the concentration of the hematopoietic progenitor cell in the pharmaceutical composition is 1×10³ cells/mL to 1×10⁷ cells/mL, preferably 1×10⁴ cells/mL to 1×10⁶ cells/mL, and more preferably 1×10⁵ cells/mL to 9.9×10⁵ cells/mL.

In fifth aspect, the present disclosure provides use of the cell according to the third aspect of the present disclosure.

Preferably, the use is use in preparing a medicament for treating and/or preventing a hematological disorder.

Preferably, provided is a method for treating a hematological disorder, comprising: administering to a subject in need thereof the hematopoietic progenitor cell according to the third aspect of the present disclosure or the pharmaceutical composition according to the fourth aspect of the present disclosure.

Preferably, the subject is a mammal, more preferably a primate, and even more preferably a human.

Preferably, the site of administration is intravenous or intramedullary in the subject.

The hematological disorder refers to a disease related to cytopathy in the blood, including but not limited to: anemia, thrombocytopenia, leukemia, lymphoma, severe aplastic anemia, multiple myeloma, or a combination thereof.

The present disclosure has the following beneficial effects:
1, the differentiation method of the present disclosure can be used for the preparation, in-vitro expansion, and maintenance of hematopoietic progenitor cells; the method features rapid and efficient preparation of hematopoietic progenitor cells, and the prepared hematopoietic progenitor cells have the ability to stably differentiate into a variety of different blood cells (including erythroid, myeloid, and lymphoid cells at the same time).
2, the differentiation method significantly improves the differentiation efficiency and the number of obtained hematopoietic progenitor cells by optimizing the culture system; the markers exhibit a high and stable expression proportion and uniformity in expression in the target cells.

### BRIEF DESCRIPTION OF THE DRAWINGS

FIG. 1 illustrates microscopic graphs of the initial stem cells according to Example 1.

FIG. 2 illustrates cytometric graphs of stemness markers in the initial stem cell according to Example 1; FIG. 2a illustrates the detection of surface stemness markers; FIG. 2b illustrates the detection of intramembrane stemness markers.

FIG. 3 illustrates microscopic graphs of the DO cells and cells at the mesoderm stage after 2 days of differentiation according to Example 1; the upper panel of FIG. 3 illustrates the morphology of cells at the start of differentiation on DO; the lower panel of FIG. 3 illustrates the morphology of cells at the mesoderm stage after two days of differentiation.

4 illustrates cytometric graphs of the mesodermal cells after two days of differentiation according to Example 1.

FIG. 5 illustrates microscopic graphs of the cells at the hemogenic endothelium stage after another 4 days of differentiation according to Example 1.

FIG. 6 illustrates cytometric graphs of the cells at the hemogenic endothelium stage after another 4 days of differentiation according to Example 1.

FIG. 7 illustrates microscopic graphs of the cells at the hematopoietic progenitor cell stage after another 6 days of differentiation according to Example 1.

FIG. 8 illustrates cytometric graphs of the cells at the hematopoietic progenitor cell stage after another 6 days of differentiation according to Example 1.

FIG. 9 illustrates graphs showing the QPCR detection results of genes associated with cells at various stages in Example 1.

FIG. 10 illustrates CFU cell morphologic graphs (CFU-E in the upper left panel, CFU-GEMM in the upper right panel, BFU-E in the lower left panel, and CFU-GM in the lower right panel) of hematopoietic progenitor cells according to Example 1.

FIG. 11 illustrates a graph showing the CFU assay results according to Examples 1 and 2.

FIG. 12 illustrates microscopic graphs of the initial stem cells according to Example 2.

FIG. 13 illustrates cytometric graphs of stemness markers in the initial stem cell according to Example 2; FIG. 13a illustrates the detection of surface stemness markers; FIG. 13b illustrates the detection of intramembrane stemness markers.

FIG. 14 illustrates microscopic graphs of the cells at the mesoderm stage after 2 days of differentiation according to Example 2.

FIG. 15 illustrates cytometric graphs of the mesodermal cells after two days of differentiation according to Example 2.

FIG. 16 illustrates microscopic graphs of the cells at the hemogenic endothelium stage after another 4 days of differentiation according to Example 2.

FIG. 17 illustrates cytometric graphs of the cells at the hemogenic endothelium stage after another 4 days of differentiation according to Example 2.

FIG. 18 illustrates a microscopic graph of the cells at the hematopoietic progenitor cell stage after another 6 days of differentiation according to Example 2.

FIG. 19 illustrates cytometric graphs of the cells at the hematopoietic progenitor cell stage after another 6 days of differentiation according to Example 2.

FIG. 20 illustrates graphs showing the QPCR detection results of genes associated with cells at various stages in Example 2.

FIG. 21 illustrates a microscopic graph of hematopoietic progenitor cells obtained by 12 days of differentiation according to Example 3.

FIG. 22 illustrates cytometric graphs of hematopoietic progenitor cells obtained by 12 days of differentiation according to Example 3.

FIG. 23 illustrates cytometric graphs of hematopoietic progenitor cells obtained by 12 days of differentiation according to Example 4.

FIG. 24 illustrates cytometric graphs of hematopoietic progenitor cells obtained by 12 days of differentiation according to Example 5.

FIG. 25 illustrates cytometric graphs of hematopoietic progenitor cells obtained by 12 days of H1-P6 cell differentiation according to Example 6.

FIG. 26 illustrates cytometric graphs of hematopoietic progenitor cells obtained by 12 days of H1-P7 cell differentiation according to Example 6.

FIG. 27 illustrates cytometric graphs of hematopoietic progenitor cells obtained by 12 days of H1-P8 cell differentiation according to Example 6.

### DETAILED DESCRIPTION

The present disclosure will be described in further detail with reference to specific examples. It will be appreciated that such examples are merely intended to illustrate the present disclosure rather than limit the scope of the present disclosure.

### Definitions

The term “pluripotent” refers to stem cells having the potential to differentiate into all cells of one or more tissues or organs, e.g., any of the three germ layers: endoderm (inner gastric wall, gastrointestinal tract, lung), mesoderm (muscles, bones, blood, urinary and genital organs), or ectoderm (epidermal tissues and nervous system).

The “pluripotent stem cell” refers to a cell capable of producing cells of all three germ layers, i.e., endoderm, mesoderm, and ectoderm. Although theoretically pluripotent stem cells can differentiate into any cell of the body, pluripotency assays are generally based on the differentiation of pluripotent cells into several cell types in each germ layer. Preferably, the pluripotent stem cell is derived from a mammal, more preferably from a primate, and even more preferably from a human. The pluripotent stem cell includes but is not limited to an embryonic stem cell and/or an induced pluripotent stem cell. Preferably, the human pluripotent stem cell (PSC) is a human embryonic stem cell (hESC) (e.g., H1, H9) and/or a human induced pluripotent stem cell (hiPSC) (e.g., WC50, IMR90). Preferably, the human embryonic stem cell is a commercially available human embryonic stem cell line. In some embodiments, the human embryonic stem cell is a stem cell isolated or obtained from a human embryo within 14 days of fertilization without in vivo development.

The term “induced pluripotent stem cell,” usually abbreviated as iPS cell or iPSC, refers to a pluripotent stem cell such as a muscle cell, neuron, epidermal cell, etc., artificially prepared from a non-pluripotent cell (usually an adult somatic cell) or a terminally differentiated cell (e.g., fibroblast, hematopoietic cell) by introducing or contacting with a reprogramming factor.

The term “embryonic stem cell,” often abbreviated as ES cell or ESC, is a pluripotent stem cell derived from an early embryo.

The term “differentiation” refers to a process by which a less specialized cell forms progeny of at least one more new specialized cell type.

The term “embryoid body,” or an embryoid or aggregate, refers to a homogeneous or heterogeneous cell cluster comprising differentiated cells, partially differentiated cells, and/or pluripotent stem cells in suspension culture. To summarize some clues inherent to in vivo differentiation, certain aspects of the present disclosure may use a three-dimensional embryoid body as an intermediate step. At the start of cell aggregation, differentiation may be initiated and cells may begin to reproduce embryonic development to a limited extent. Despite trophectoderm tissues, they may develop almost all other types of cells present in an organism. The present disclosure can further promote the differentiation of hematopoietic progenitor cells after the formation of embryoid bodies.

The term “hematopoietic progenitor cell” refers to a hematopoietic progenitor cell formed by the induction of directed differentiation from a pluripotent stem cell, as described in the first aspect of the present disclosure. The hematopoietic progenitor cells of the present disclosure are hematopoietic progenitor cells having the ability to differentiate into erythroid, myeloid, and lymphoid lineages.

The term “basal medium” refers to a chemically-defined medium, including but not limited to, basal cell culture media such as Iscove's modified Dulbecco's medium (IMDM), Eagle's Basal Medium (BME), Eagle MEM, DMEM, Ham, RPMI1640, and Fischer medium.

The term “vitamin C” encompasses vitamin C or salts or derivatives thereof of various forms.

The term “glutamine” refers to L-glutamine, a coding amino acid in protein synthesis, a non-essential amino acid for mammals, and an essential additive for cell culture in the present disclosure.

The term “SB431542” also encompasses SB431542 and salts thereof, especially pharmaceutically acceptable salts thereof.

The term “CHIR99021” encompasses CHIR99021 and salts thereof, especially pharmaceutically acceptable salts thereof.

The term “Y-27632” also encompasses Y-27632 and salts thereof, especially pharmaceutically acceptable salts thereof. A preferred pharmaceutically acceptable salt is Y-27632 2HCL.

The term “monothioglycerol” or MTG is a necessary reductant for stem cell culture, equivalent to β-mercaptoethanol.

The “bone morphogenetic protein-4” or BMP4 has regulatory effects on the proliferation and differentiation of various cells during embryonic development.

The term “vitamin A-free B27 additive” refers to a customized B-27® additive free of vitamin A. Vitamin A (retinol) can be converted to retinoic acid, which induces the differentiation of stem cells into nerve cells. Formulations free of vitamin A are ideal for stem cell culture.

Those of ordinary skills in the art can employ, treat, administer, etc., the hematopoietic progenitor cells using conventional methods. For example, before the release or use of each batch of hematopoietic progenitor cells, the cells must pass the sterility assay, endotoxin assay, mycoplasma assay, and DNA identity assay. Every release of cells shall meet the criteria of viability ≥95% and cell purity (positive indicators ≥95%, and negative indicators <2%). The results of acute toxicity assay and allergy assay of the hematopoietic progenitor cells should be negative.

### Detection Marker for Cells

The surface stemness markers of the stem cell of the present disclosure are SSEA4, TRA-1-81, and TRA-1-60; intramembrane stemness markers are Nanog, Oct4, and Sox2. The marker for assay of cells at the mesoderm stage is KDR. The markers for assay of cells at the hemogenic endothelium stage are CD31, CD34, CD43, and CD309. The hematopoietic progenitor cell prepared by the method of the present disclosure can be confirmed by the assay of cell surface antigens CD34, CD43, CD45, CD90, and CD117.

The CD34 antigen is a highly glycosylated single-pass transmembrane protein that is selectively expressed on the surface of human hematopoietic stem cells (HSCs), hematopoietic progenitor cells (HPCs), and vascular endothelial cells (ECs). In the present disclosure, in the total cell population differentiated in step (4) without purification and/or enrichment procedures, the proportion of the hematopoietic progenitor cells expressing CD34 is preferably ≥90% in the total cell population.

The KDR antigen, or CD309, is a vascular endothelial growth factor (VEGF) receptor that is widely expressed in a variety of mesodermal tissues during development and in vascular endothelial cells at the embryonic stage. In-vitro and in-vivo data show that KDR is critical for the development of vascular endothelial cells and hematopoietic cells. In the present disclosure, in the total cell population differentiated in step (4) without purification and/or enrichment procedures, the proportion of the hematopoietic progenitor cells expressing KDR is preferably ≥90% in the total cell population.

The CD43 antigen, also known as leukosialin or sialophorin, is a glycoprotein encoded by the SNP gene and expressed on the surface of most blood leukocytes such as B cells, T cells, NK cells, and granulocytes. In the present disclosure, hematopoietic progenitor cells were differentiated from iPSCs by means of the CD43-positive feature. In the present disclosure, in the total cell population differentiated in step (4) without purification and/or enrichment procedures, the proportion of the hematopoietic progenitor cells expressing CD43 is preferably ≥90% in the total cell population.

The CD45 antigen is composed of a group of transmembrane proteins with similar structures and large molecular weights, and is widely present on the surface of leucocytes. The cytoplasmic region of the antigen has the function of protein tyrosine phosphatase and can dephosphorylate tyrosines on substrates P56lck and P59fyn and activate the substrates, thus playing an important role in the signaling of cells. CD45 is a surface marker of mature blood precursor cells. In the present disclosure, in the total cell population differentiated in step (4) without purification and/or enrichment procedures, the proportion of the hematopoietic progenitor cells expressing CD45 is preferably ≥90% in the total cell population.

The purity and degree of differentiation of the hematopoietic progenitor cells of the present disclosure can be measured using conventional methods, such as flow cytometry. During the detection, different specific antibodies specific to corresponding cell surface antigens are added, wherein the antibodies can be intact monoclonal or polyclonal antibodies, and can also be antibody fragments with immunological activity, such as Fab′ or (Fab)2 fragments; single chain Fv molecules (scFVs); or chimeric antibodies. The added antibodies interact with the antigens on the cell surface for a period of time, and the cells can be automatically analyzed and/or sorted using a flow cytometer.

Experimental methods without specified conditions in the following examples are generally conducted in conventional conditions, or conditions recommended by the manufacturer. The materials, reagents, and the like used in the examples are commercially available reagents and materials unless otherwise specified.

The following Table 1 lists the sources of the reagents employed in the present disclosure:

TABLE 1
Sources of reagents

| Reagent | Company |
|---|---|
| hiPSCs (XS-iPS) | XellSmart |
| H1 cell/H9 cell | ZQXZBIO |
| DMEM/F12 medium | Gibco |
| E8 medium | Gibco |
| RPM1640 medium | Gibco |
| IMDM medium | Gibco |
| Accutase | 4A Biotech |
| DPBS | BasalMedia |
| BSA | Sigma |
| B27-supplement without vtaminA | Gibco |
| Y27632 | Selleck |
| RelesR | Stem cell |
| NEAA | Gibco |
| Glutamax | Gibco |
| Vitamin C | Sigma |
| BMP4 | Peprotech |
| CHIR-99021 | Selleck |
| VEGF-165 | Sino Biological |
| FGF2 | Origene |
| SB431542 | Selleck |
| SCF | Peprotech |
| N-acetyl-L-cysteine | Sigma |
| Minocycline hydrochloride | Selleck |
| Penicillin-streptomycin | Gibco |
| Monothioglycerol | Sigma |

### Example 1. Preparation of Hematopoietic Progenitor Cells

The pluripotent stem cells used were ESCs, specifically, H1 cells.

### 1. Preparation of Hematopoietic Progenitor Cells

1.1 Pluripotent Stem Cell Passage and Differentiation (H1 Cell Culture Conditions: Adherent Culture in Six-Well Plates without Trophoblast; H1 Cell Differentiation: Low-Adherence Suspension Culture in Six-Well Plates)
D-1 medium: E8 μMedium+10 μM Y27632.

Pluripotent stem cells at a confluence of about 80% were taken from the incubator, and the culture medium was discarded. The cells were washed once with DPBS, and DPBS was discarded. Accutase was added at 0.5 mL/well, and the plates were incubated for 2 min in a carbon dioxide incubator at 37° C. After 2 min, DMEM/F12 culture medium was added to stop the enzymatic reaction in each well. The cells were detached by pipetting, transferred into a centrifuge tube, and centrifuged at 1200 r for 5 min. The supernatant was discarded before 1 mL of E8 culture medium was added to resuspend the cells. The cells were counted by AO/PI staining. A desired amount of cells were added to the D-1 medium, and the cells were plated in low-adherence six-well plates at 8×10⁴ cells/well. The day was designated as D-1 of differentiation. Cells were shaken vertically and horizontally (cross method), and cultured in an incubator at 37° C./5% CO₂ for 20-24 hours.

### 1.2 Stage I: Induced Mesoderm Differentiation (D0-D1)

Stage I medium: RPM1640 medium+2% of vitamin A-free B27 additive+1% of non-essential amino acids+1% glutamine+50 μg/mL of vitamin C+20 ng/mL of BMP4+3 μM of CHIR-99021.

The cells seeded on D-1 were taken from the incubator and observed under a microscope (photographing and recording, wherein the cells were round). The low-adherence six-well plates were obliquely placed, and part of the supernatant was discarded, leaving about 500 μL of the supernatant. The Stage I culture medium was added at 2 mL/well. The plates were gently shaken and returned to the incubator. The cells were incubated for two days of differentiation, collected, and detected for KDR+ expression proportion in endothelial cells by flow cytometry. The culture time was 42-48 hours.

### 1.3 Stage II: Induced Hemogenic Endothelium Differentiation (D2-D5)

Stage II medium: RPM1640 Medium+2% of vitamin A-free B27 additive+1% of non-essential amino acids+1% of glutamine+50 μg/mL of vitamin C+5 ng/mL of BMP4+50 ng/mL of VEGF-165+50 ng/mL of FGF2+10 μM of SB431542.

On D2, the cells were taken from the incubator and observed under a microscope (photographing and recording). The low-adherence six-well plates were obliquely placed, and part of the supernatant was discarded, leaving about 500 μL of the supernatant in each well. The Stage II culture medium was added at 2 mL/well. The plates were gently shaken and returned to the incubator. On D4, the cells were taken from the incubator. The low-adherence six-well plates were obliquely placed, and the supernatant was discarded. The Stage II culture medium was added at 2 mL/well. The plates were shaken and returned to the incubator. On D6, the cells were collected and determined for the hemogenic endothelium phenotype expression proportion by flow cytometry.

### 1.4 Stage III: Induced Hematopoietic Progenitor Cell Differentiation (D6-D12)

Stage III medium: IMDM Medium+2% of vitamin A-free B27 additive+1% of NEAA+1% of GlutaMax+50 μg/mL of vitamin C+5 ng/mL of BMP4+10 ng/mL of VEGF-165+20 ng/mL of SCF+30 μM of NAC (N-acetyl-L-cysteine)+2 μM of minocycline hydrochloride.

On D6, the cells were taken from the incubator and observed under a microscope (photographing and recording). The low-adherence six-well plates were obliquely placed, and part of the supernatant was discarded, leaving about 500 μL of the supernatant in each well. The Stage III culture medium was added at 2 mL/well. The plates were gently shaken and returned to the incubator. On D8, the cells were taken from the incubator. The low-adherence six-well plates were obliquely placed, and the medium was discarded. The Stage III culture medium was added at 2 mL/well. The plates were gently shaken and returned to the incubator. On D10, half of the medium was replaced in the same manner as D8. On D12, the cells were collected by centrifugation (1500 rpm, 5 min), and the supernatant was discarded. The cells were resuspended in 5 mL of Stage III culture medium and filtered through a 40-μm sieve. The filtered cells were centrifuged (1500 rpm, 5 min), and the supernatant was discarded. The cells were resuspended in 1 mL of Stage III culture medium, counted, and determined for the marker ratios of CD34+, CD45+, etc., on the surface of the hematopoietic progenitor cells by flow cytometry.

### 2. Detection of Cells at Different Stages

The flow cytometry detection procedures are as follows:
1) The cells were collected and centrifuged at 1500 rpm for 3 minutes;
2) The supernatant was discarded, and the cells were resuspended in PBS, filtered through a 40-mesh sieve, counted via AO/PI staining, and centrifuged for 5 minutes at 1500 rpm;
3) After discarding the supernatant, PBS was added, and the cells were centrifuged for 3 minutes at 1500 rpm before PBS was discarded;
4) The cells were resuspended in 200 μL of PBS containing 0.5% of BSA, antibodies for flow cytometry were added as required away from light, and the system was mixed and let stand in a 4° C. refrigerator for 30 min;
5) After 30 minutes, the cells were taken from the 4° C. refrigerator before the addition of 1 mL of 0.5% BSA and centrifuged at 1500 rpm for 5 minutes;
6) The supernatant was discarded, and the cells were washed with 500 μL of PBS and centrifuged at 1500 rpm for 5 minutes;
7) The supernatant was discarded, and the cells were resuspended in 200 μL of PBS and loaded on a flow cytometer for detection (NovoCyte 3000, sample injector: NS200, manufacturer: Agilent Technologies).

### 2.1 Initial Stem Cells

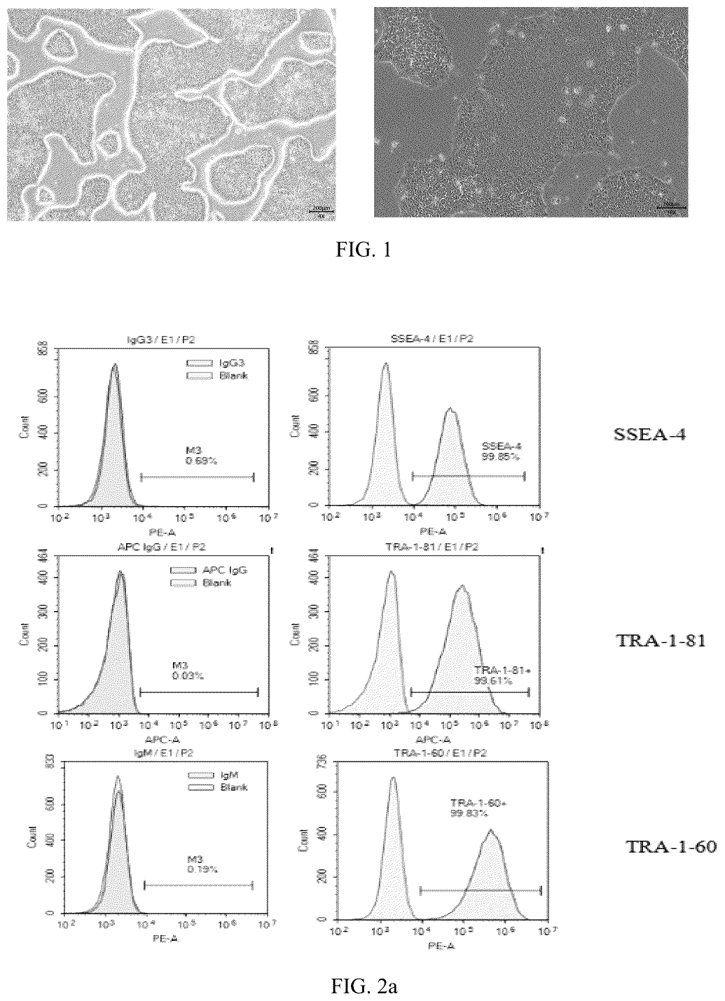

The initial stem cells of Example 1 were collected and observed under a microscope, and the morphology thereof is shown in FIG. 1. The left panel of FIG. 1 shows the cell morphology at 4×, and the right panel of FIG. 1 shows the cell morphology at 10×.

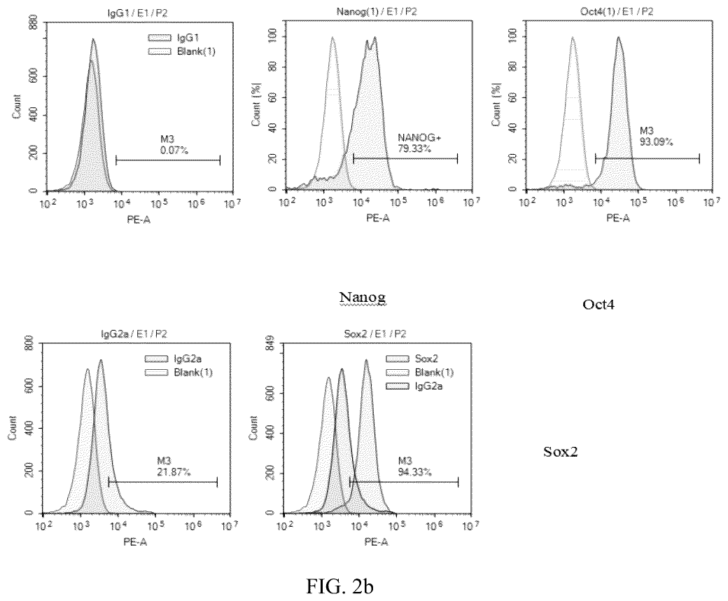

The initial stem cells were subjected to flow cytometric analysis to detect their stemness markers. The results for surface stemness marker (SSEA4, TRA-1-81, TRA-1-60) detection are shown in FIG. 2a, and the results for intramembrane stemness marker (Nanog, Oct4, Sox2) detection are shown in FIG. 2b. As shown in FIG. 2a, the expression proportion was 99.85% for SSEA4, 99.61% for TRA-1-81, and 99.83% for TRA-1-60; as shown in FIG. 2b, the expression proportion was 79.33% for Nanog, 93.09% for Oct4, and 94.33% for Sox2. This indicates that the initial embryonic stem cells did not exhibit a tendency to differentiate and were normal and acceptable ESCs.

### 2.2 Stage I: Mesoderm Stage (D0-D1)

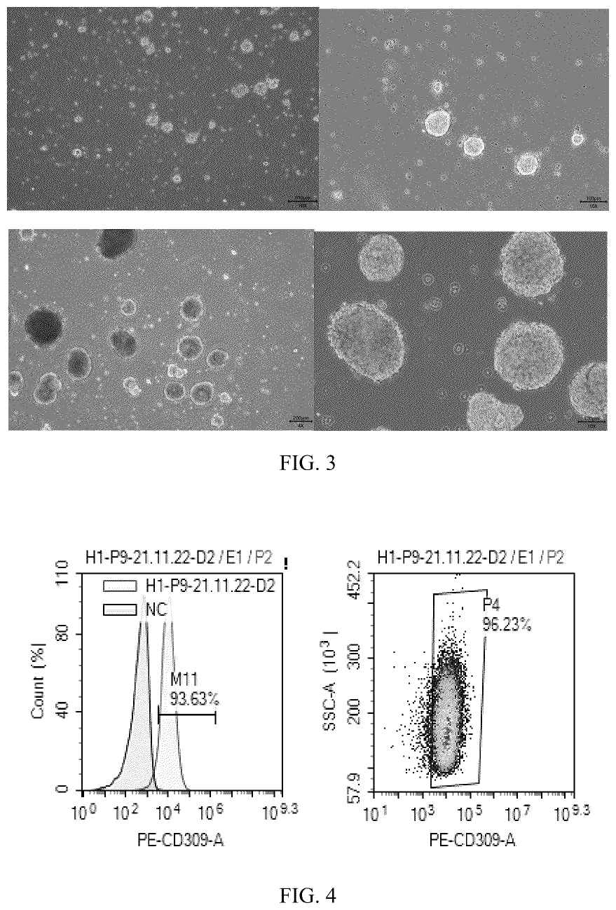

The D0 cells before differentiation and D2 cells that had not entered the hemogenic endothelium stage two days after differentiation in Example 1 were collected and observed under a microscope, and the morphology is shown in FIG. 3. The upper left panel of FIG. 3 shows the morphology (4×) of ES cells on D0 at the start of differentiation, and the upper right panel of FIG. 3 shows the morphology (10×) of ES cells on D0 at the start of differentiation. The lower left panel of FIG. 3 shows the mesodermal cell morphology (4×) of ES cells after two days of differentiation, and the lower right panel of FIG. 3 shows the mesodermal cell morphology (10×) of ES cells after two days of differentiation. As shown in FIG. 3, the cells at the mesoderm stage after two days of differentiation had a diameter of about 100-200 μm.

The cells at the mesoderm stage after two days of differentiation were analyzed by flow cytometric analysis for their KDR+(CD309) expression. As shown in FIG. 4, for the cells at the mesoderm stage (Stage I) after two days of differentiation, the proportion of KDR+ cells was up to 96.23%, indicating that the differentiated cells were at the mesoderm-endothelium stage.

### 2.3 Stage II: Induced Hemogenic Endothelium Differentiation Stage (D2-D5)

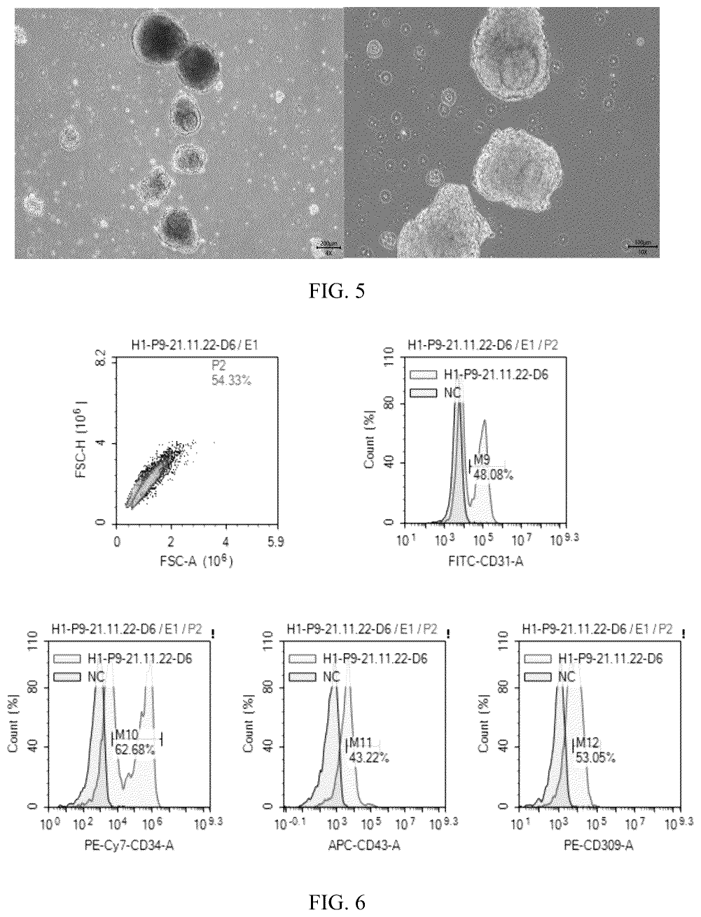

The D6 cells that had undergone another 4 days of differentiation in the induced hemogenic endothelium differentiation stage (Stage II) but had not entered the induced hematopoietic progenitor cell differentiation stage in Example 1 were collected and observed under a microscope, and the morphology thereof is shown in FIG. 5. The left panel of FIG. 5 shows the cell morphology at 4×, and the right panel shows the cell morphology at 10×. As shown in FIG. 5, the cells at the hemogenic endothelium stage after 6 days of differentiation had a diameter of about 150-300 μm.

The cells at the induced hemogenic endothelium differentiation stage (Stage II) after another 4 days of differentiation were subjected to flow cytometry analysis to determine the expression of markers CD31, CD34, CD43, and KDR (CD309). As shown in FIG. 6, the positive expression proportion was 48.08% for FITC-CD31, 62.68% for PE-Cy7-CD34, 43.22% for APC-CD43, and 53.05% for PE-CD309.

### 2.4 Stage III: Induced Hematopoietic Progenitor Cell Differentiation (D6-D12)

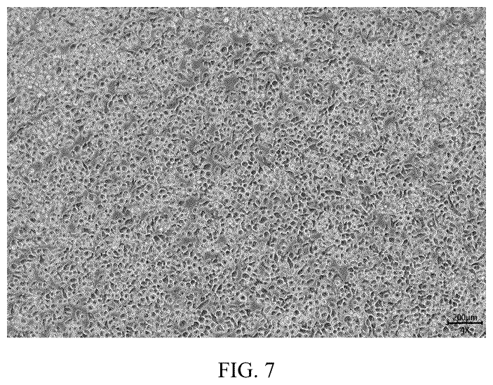

The D12 cells that had undergone another 6 days of differentiation in the induced hematopoietic progenitor cell differentiation stage (Stage III) in Example 1 were collected and observed under a microscope, and the morphology thereof is shown in FIG. 7. FIG. 7 shows the cell morphology at 4×. The hematopoietic progenitor cells after 12 days of differentiation were approximately 8 μm in size and were individual mononuclear cells with a round shape and a large nucleus.

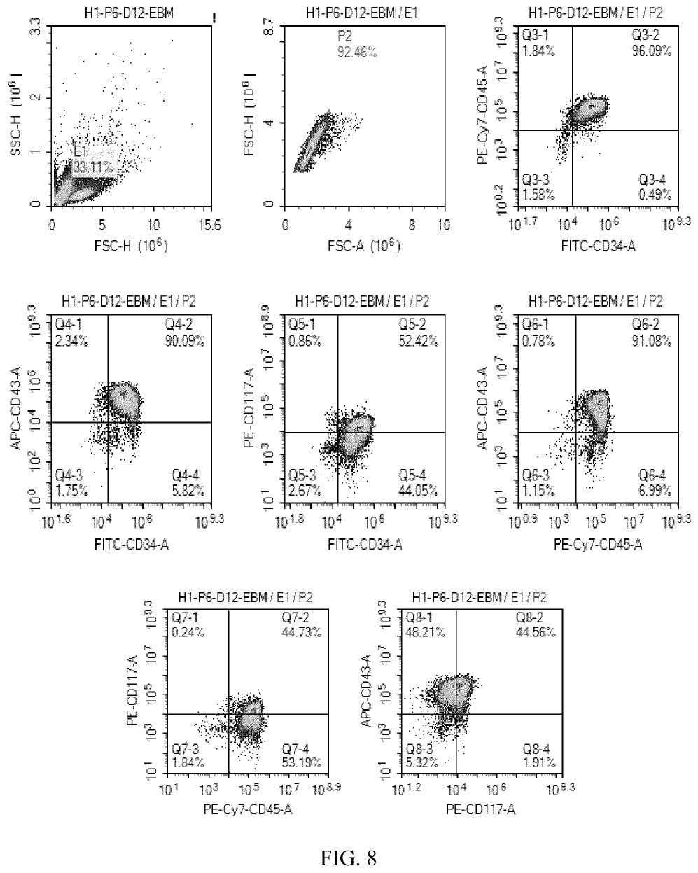

The hematopoietic progenitor cells at Stage III after another 6 days of differentiation were subjected to flow cytometry analysis to determine the expression of markers. As shown in FIG. 8, the flow cytometric results on D12 of differentiation are as follows: the proportion was 96.09% for CD34+/CD45+, 90.09% for CD34+/CD43+, and 91.08% for CD45+/CD43+. 3. Counting of hematopoietic progenitor cells
(1) The D12 cells were taken from the incubator and observed under a microscope (photographing and recording);
(2) The cells were transferred into an ultra clean bench, gently shaken, and transferred into a centrifuge tube;
(3) The cells in the centrifuge tube were centrifuged at 1500 rpm for 5 min;
(4) The supernatant was discarded, the cells were resuspended in 3 mL of stage III culture medium, and the cell suspension was then filtered through a 40-μm sieve (to remove undifferentiated EB cells);
(5) The cell suspension filtrate was centrifuged at 1500 rpm for 5 min;
(6) The supernatant was discarded, the cells were resuspended in 1 mL of stage III culture medium, 15 μL of the cell suspension was mixed with 15 μL of AO/PI dye, and 20 μL of the mixture was transferred to a cell counting plate and loaded for counting.

The H1 cell count results on D12 of differentiation are: AO/PI cell count: 2.08×10⁶ (cell count in each well of the six-well plate); viability: 90.25%.

### 4. QPCR Assay of Gene Expression Related to Differentiated Hematopoietic Cells at Various Stages

Reagent: FastPure Cell/Tissue Total RNA Isolation Kit V2 (Vazyme), HiScript III RT SuperMix for qPCR (+ gDNA wiper) (Vazyme), ChamQ Universal SYBR qPCR Master Mix (Vazyme); fluorescent quantitative PCR system Light Cycle 480 II 96/384 (Roche).
(1) The sequences of genes FLI1, SOX17, GATA1, CDX2, CXCL12, GATA2, RUNX1, KITLG, GFI1B, GFI1, MYB, HOXA10, HOXA5, HOXA6, and HOXA9 were retrieved on NCBI, and the primers were designed by software primer5 and constructed by GENECHEM;
(2) RNAs were extracted using FastPure Cell/Tissue Total RNA Isolation Kit V2;
(3) The extracted total RNAs were subjected to reverse transcription using HiScript III RT SuperMix for qPCR (+ gDNA wiper): 15 min at 37° C.; 5 sec at 85° C.;
(4) qPCR assays using ChamQ Universal SYBR qPCR Master Mix: 180 μL of DNase/RNase Free ultrapure water was added to the cDNA of the reverse transcription 20 μL system to give a qPCR template; the reaction system was prepared, containing 5 μL of 2× ChamQ Universal SYBR qPCR Master Mix, 0.2 μL of Primer 1 (10 μM), 0.2 μL of Primer 2 (10 μM), X μL of Template cDNA, and 10 μL of ddH₂O To as per 10 μL of the reaction system; the samples were loaded on a Roche QPCR system for assay. The primer sequences are shown below (column 2 is the upstream primer, column 3 is the downstream primer, and the last column is the amplified bp number).

| | | | Product |
|---|---|---|---|
| | | | length |
| Gene | Forward | Reverse | (bp) |
| CXCL12 | ATTCTCAACACTCCAAACTGT | ACTTTAGCTTCGGGTCAATGC | 88 |
| | GC (SEQ ID NO: 1) | (SEQ ID NO: 2) | |
| FLI1 | CAGCCCCACAAGATCAACCC | CACCGGAGACTCCCTGGAT | 111 |
| | (SEQ ID NO: 3) | (SEQ ID NO: 4) | |
| GATA1 | CTGTCCCCAATAGTGCTTATG | GAATAGGCTGCTGAATTGAGG | 88 |
| | G (SEQ ID NO: 5) | G (SEQ ID NO: 6) | |
| GATA2 | GCAACCCCTACTATGCCAACC | CAGTGGCGTCTTGGAGAAG | 212 |
| | (SEQ ID NO: 7) | (SEQ ID NO: 8) | |
| GFI1 | CCGCGCTCATTTCTCGTCA | ACGGAGGGAATAGTCTGGTCC | 81 |
| | (SEQ ID NO: 9) | (SEQ ID NO: 10) | |
| GFI1B | GCAGGAAGATGAACCGCTCT | CCAGGCACTGGTTTGGGAA | 103 |
| | (SEQ ID NO: 11) | (SEQ ID NO: 12) | |
| HOXA10 | CTCGCCCATAGACCTGTGG | GTTCTGCGCGAAAGAGCAC | 151 |
| | (SEQ ID NO: 13) | (SEQ ID NO: 14) | |
| HOXA5 | AACTCATTTTGCGGTCGCTAT | TCCCTGAATTGCTCGCTCAC | 89 |
| | (SEQ ID NO: 15) | (SEQ ID NO: 16) | |
| HOXA6 | TCCCGGACAAGACGTACAC | CGCCACTGAGGTCCTTATCA | 123 |
| | (SEQ ID NO: 17) | (SEQ ID NO: 18) | |
| HOXA9 | TACGTGGACTCGTTCCTGCT | CGTCGCCTTGGACTGGAAG | 153 |
| | (SEQ ID NO: 19) | (SEQ ID NO: 20) | |
| MYB | GAAAGCGTCACTTGGGGAAAA | TGTTCGATTCGGGAGATAAT | 122 |
| | (SEQ ID NO: 21) | TGG (SEQ ID NO: 22) | |
| RUNX1 | CTGCCCATCGCTTTCAAGGT | GCCGAGTAGTTTTCATCATT | 92 |
| | (SEQ ID NO: 23) | GCC (SEQ ID NO: 24) | |
| CDX2 | GACGTGAGCATGTACCCTAGC | GCGTAGCCATTCCAGTCCT | 215 |
| | (SEQ ID NO: 25) | (SEQ ID NO: 26) | |
| SOX17 | GTGGACCGCACGGAATTTG | GGAGATTCACACCGGAGTCA | 94 |
| | (SEQ ID NO: 27) | (SEQ ID NO: 28) | |
| KITLG | AATCCTCTCGTCAAAACTGAA | CCATCTCGCTTATCCAACAA | 163 |
| | GG (SEQ ID NO: 29) | TGA (SEQ ID NO: 30) | |
| GAPDH | GGAGCGAGATCCCTCCAAAAT | GGCTGTTGTCATACTTCTCA | 197 |
| | (SEQ ID NO: 31) | TGG (SEQ ID NO: 32) | |

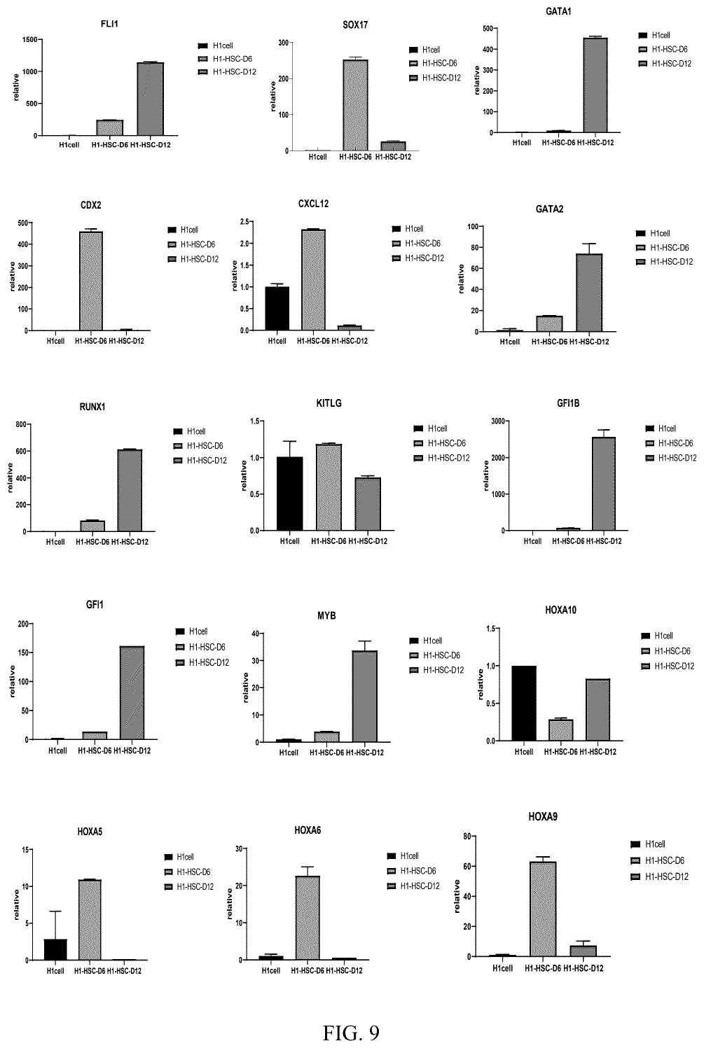

The results of QPCR assay of the genes involved in the initial cells, D6 cells, and hematopoietic progenitor cells obtained at the end of differentiation are shown in FIG. 9. FIG. 9 illustrates the assay results of the following genes: FLI1, SOX17, GATA1, CDX2, CXCL 12, GATA2, RUNX1, KITLG, GFI1B, GFI1, MYB, HOXA10, HOXA5, HOXA6, and HOXA9. It can be seen that the genes associated with the hematopoietic cells were highly expressed.

### 5. Colony-Forming Unit (CFU) Assay

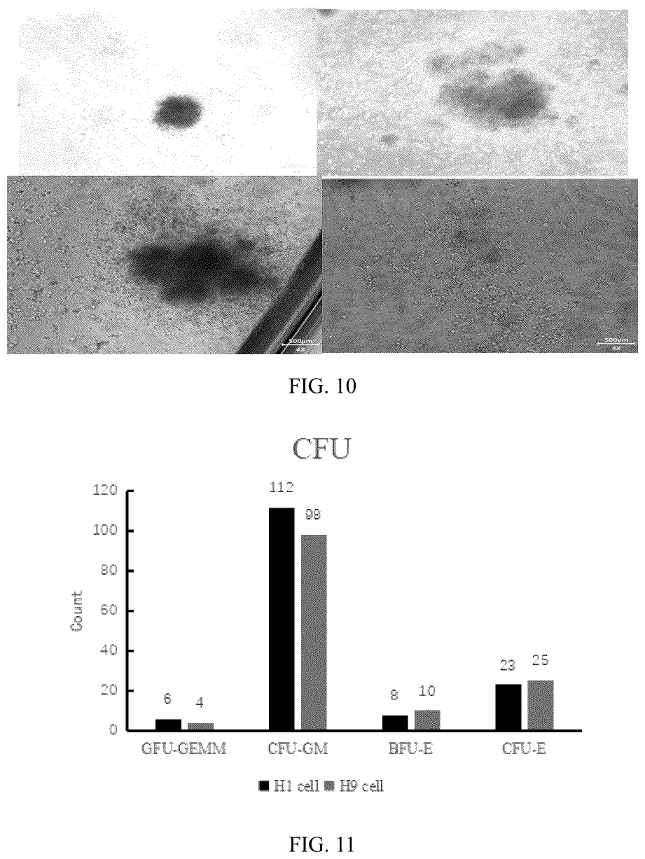

The colony-forming unit (CFU) assay is a standard for the in-vitro detection of hematopoietic stem/progenitor cell functionality. CFU colonies are generally classified into: erythroid colony-forming units (CFU-E), erythroid burst-forming units (BFU-E), granulocyte/macrophage (CFU-GM), and cell mix colony-forming units (CFU-GEMM). The specific procedures are as follows:
1. The required MethoCult® culture medium (purchased from Gibco) was thawed at 4° C. or at room temperature overnight
2. The culture medium was dispensed into 15-mL centrifuge tubes (3.3 mL/tube) using a 16 gauge blunt-needle syringe
3. Preparation of cells: The cells at the end of differentiation were collected and counted on a cell counter. 10,000 cells were resuspended in 0.3 mL of IMDM+2% of FBS, and the cell suspension was seeded on MethoCult® culture medium (in a ratio of 1:10)
4. The centrifuge tube was vortexed vigorously and let stand for 5 min to dissipate the bubbles.
5. The mixture of MethoCult® culture medium and the cells was transferred to a 6-well plate using a sterile 10-mL syringe equipped with a 16 gauge blunt needle at 3 mL/well in triplicate. The mixture of each tube was transferred with a separated set of blunt needles and syringes. The culture plate was gently inclined and rotated to evenly overspread the medium in the culture plate. The remaining blank wells and the gaps of the six-well plate were filled with PBS.
6. The 6-well plate was placed in a CO₂ incubator and incubated for 14 days.
7. After 14 days of culture, the colonies were counted. The assay results are shown in FIG. 10 (4×) and FIG. 11. The results show that the hematopoietic progenitor cells obtained in Example 1 have the ability to form CFU-E, BFU-E, CFU-GM, and CFU-GEMM, and that the hematopoietic progenitor cells obtained in Example 1 possess high stemness, good quality, and the ability to differentiate into various blood cells such as erythroid and myeloid cells.

### Example 2. Preparation of Hematopoietic Progenitor Cells

The hematopoietic progenitor cells in Example 2 were prepared as in Example 1, except that the embryonic stem cells used were H9 cells.

### 2. Detection of Cells at Different Stages

The cells of Example 2 were subjected to flow cytometry at each stage according to the method in Example 1.

### 2.1 Initial Stem Cells

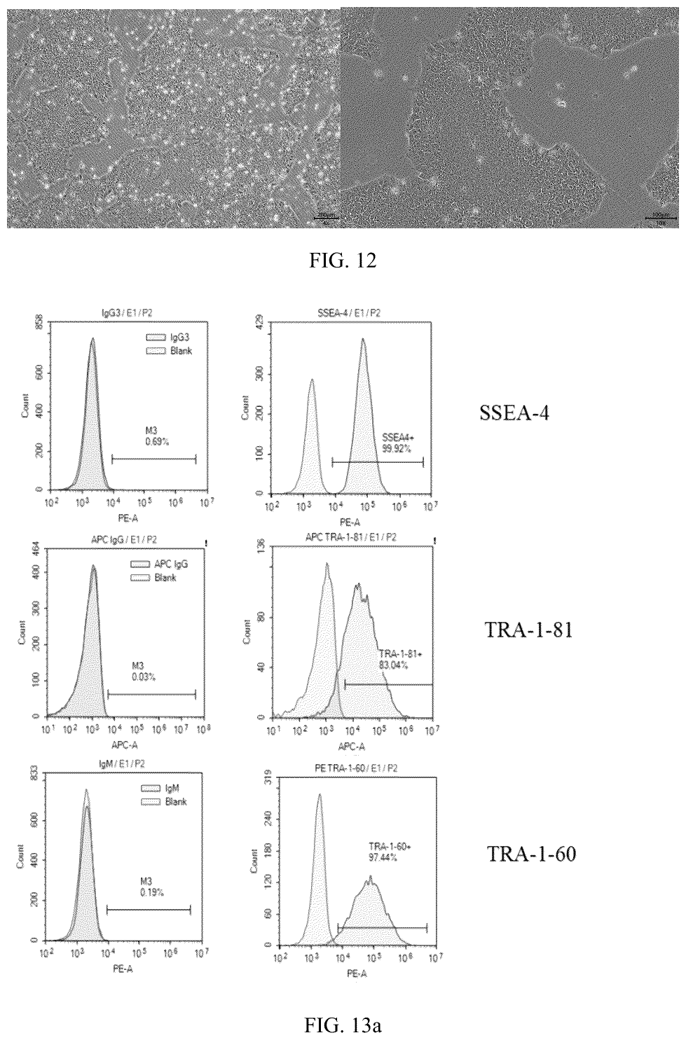

The initial stem cells of Example 2 were collected and observed under a microscope, and the morphology thereof is shown in FIG. 12. The left panel of FIG. 12 shows the cell morphology at 4×, and the right panel of FIG. 12 shows the cell morphology at 10×.

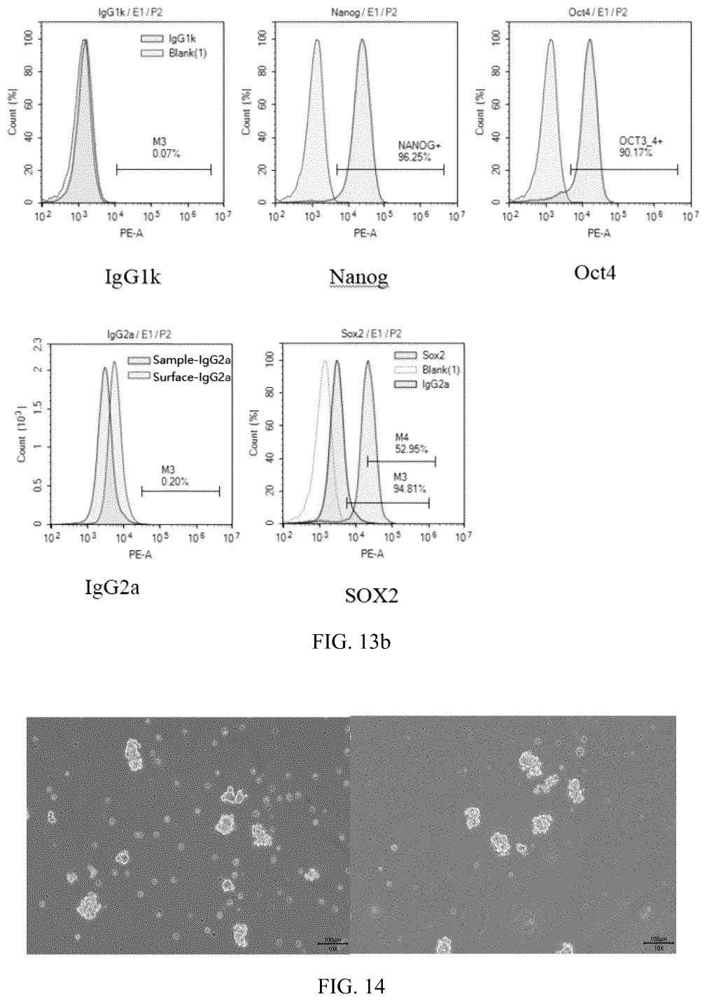

The initial stem cells of Example 2 were subjected to flow cytometric analysis to detect their stemness markers according to the method in Example 1. The results for surface stemness marker (SSEA4, TRA-1-81, TRA-1-60) detection are shown in FIG. 13a, and the results for intramembrane stemness marker (Nanog, Oct4, Sox2) detection are shown in FIG. 13b. FIG. 13 indicates that the initial embryonic stem cells did not exhibit a tendency to differentiate and were normal and acceptable ESCs.

### 2.2 Stage I: Mesoderm Stage (D0-D1)

The D2 (mesodermal) cells that had not entered the hemogenic endothelium stage two days after differentiation in Example 2 were collected and observed under a microscope, and the morphology is shown in FIG. 14. The left and right panels of FIG. 14 both show the mesodermal cell morphology (10×) of H9 cells after two days of differentiation.

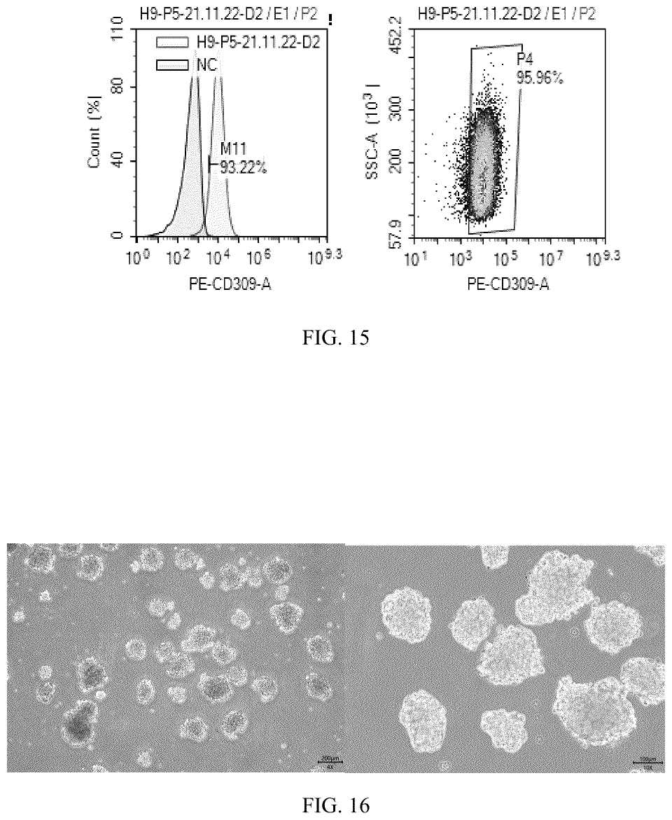

The cells at the mesoderm stage after two days of differentiation were analyzed by flow cytometric analysis for their KDR+(CD309) expression. As shown in FIG. 15, for the cells at the mesoderm stage (Stage I) after two days of differentiation, the proportion of KDR+ cells was up to 95.96%, indicating that the differentiated cells were at the mesoderm-endothelium stage.

### 2.3 Stage II: Induced Hemogenic Endothelium Differentiation Stage (D2-D5)

The D6 cells that had undergone another 4 days of differentiation in the induced hemogenic endothelium differentiation stage (Stage II) but had not entered the induced hematopoietic progenitor cell differentiation stage in Example 2 were collected and observed under a microscope, and the morphology thereof is shown in FIG. 16. The left panel of FIG. 16 shows the cell morphology at 4×, and the right panel shows the cell morphology at 10×. As shown in FIG. 16, the cells at the hemogenic endothelium stage after 6 days of differentiation had a diameter of about 150-300 μm.

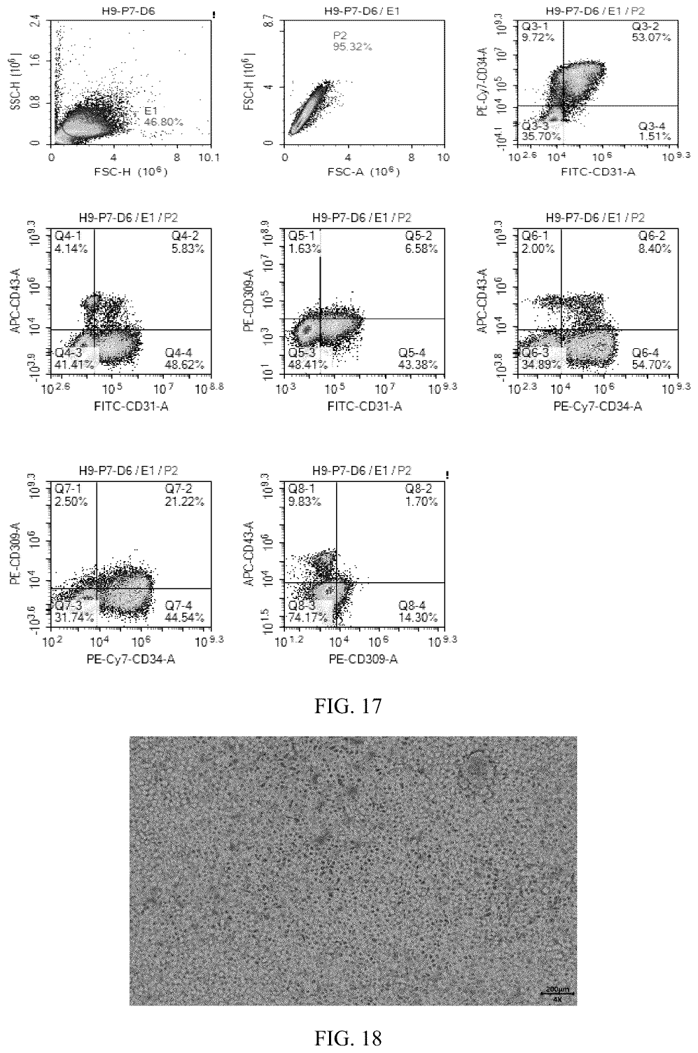

The cells at the induced hemogenic endothelium differentiation stage (Stage II) after another 4 days of differentiation were subjected to flow cytometry analysis to determine the expression of markers CD31, CD34, CD43, and KDR (CD309), as shown in FIG. 17.

### 2.4 Stage III: Induced Hematopoietic Progenitor Cell Differentiation (D6-D12)

The D12 cells that had undergone another 6 days of differentiation in the induced hemogenic endothelium differentiation stage (Stage III) in Example 2 were collected and observed under a microscope, and the morphology thereof is shown in FIG. 18. FIG. 18 shows the cell morphology at 4×. The hematopoietic progenitor cells after 12 days of differentiation were approximately 8 μm in size and were individual mononuclear cells with a round shape and a large nucleus.

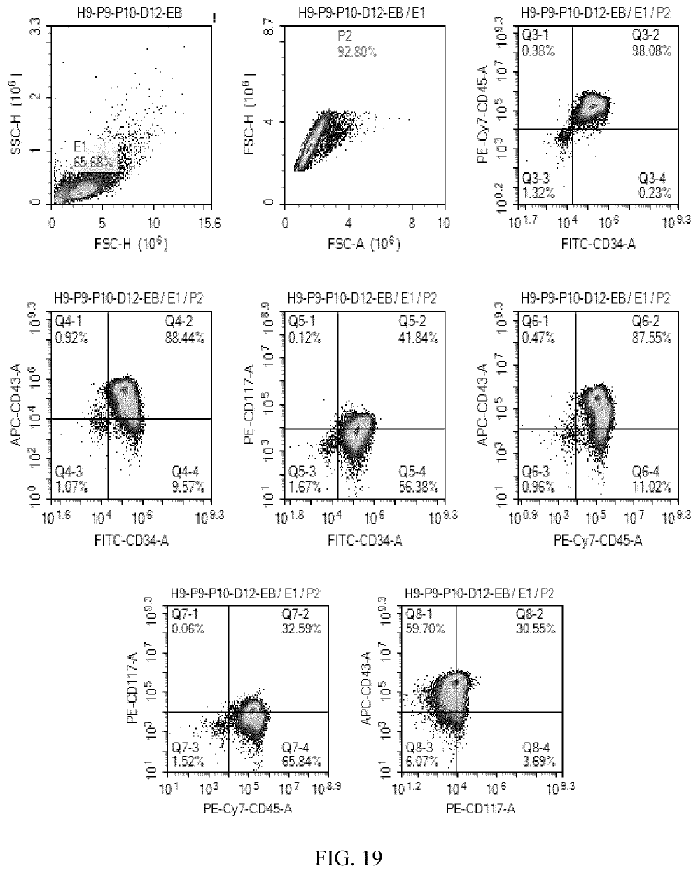

The hematopoietic progenitor cells at Stage III after another 6 days of differentiation were subjected to flow cytometry analysis to determine the expression of markers. As shown in FIG. 19, the flow cytometric results on D12 of differentiation are as follows: the proportion was 98.08% for CD34+/CD45+, 88.44% for CD34+/CD43+, and 87.55% for CD45+/CD43+.

### 3. Counting of Hematopoietic Progenitor Cells

The target cells finally obtained in Example 2 were subjected to counting according to the method in Example 1.

The H9 cell count results on D12 of differentiation are: AO/PI cell count: 1.8×10⁶ (cell count in each well of the six-well plate); viability: 91.38%.

### 4. QPCR Assay of Gene Expression Related to Differentiated Hematopoietic Cells at Various Stages

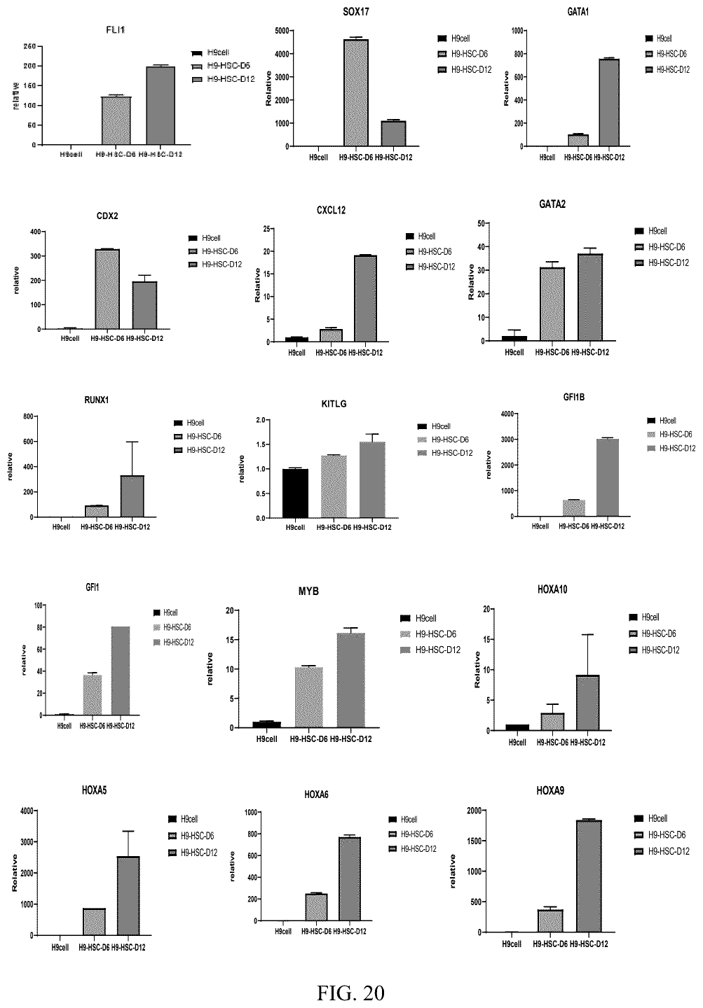

According to the method in Example 1, the results of QPCR assay of the genes involved in the initial cells, D6 cells, and hematopoietic progenitor cells obtained at the end of differentiation in Example 2 are shown in FIG. 20. FIG. 20 illustrates the assay results of the following genes: FLI1, SOX17, GATA1, CDX2, CXCL12, GATA2, RUNX1, KITLG, GFI1B, GFI1, MYB, HOXA10, HOXA5, HOXA6, and HOXA9. It can be seen that the genes associated with the hematopoietic cells were highly expressed.

### 5. Colony-Forming Unit (CFU) Assay

The hematopoietic progenitor cells obtained at the end of differentiation in Example 2 were tested for colony-forming unit according to the method in Example 1.

The assay results for the erythroid colony-forming unit (CFU-E), erythroid burst-forming unit (BFU-E), granulocyte/macrophage (CFU-GM), and cell mix colony-forming unit (CFU-GEMM) are shown in FIG. 11. The results show that the hematopoietic progenitor cells obtained in Example 2 have the ability to form CFU-E, BFU-E, CFU-GM, and CFU-GEMM, and that the hematopoietic progenitor cells obtained in Example 2 possess high stemness, good quality, and the ability to differentiate into various blood cells such as erythroid and myeloid cells. The CFU assay results of Examples 1 and 2 are shown in Table 2.

TABLE 2
CFU assay results of Examples 1 and 2

| CFU | H1 cell | H9 cell |
|---|---|---|
| CFU-GEMM | 6 | 4 |
| CFU-GM | 112 | 98 |
| BFU-E | 8 | 10 |
| CFU-E | 23 | 25 |

### Example 3. Preparation of Hematopoietic Progenitor Cells

The procedures of this example are substantially the same as those of Example 1, except that hiPSCs (from XellSmart, Cat. No. XS-iPS) were used.

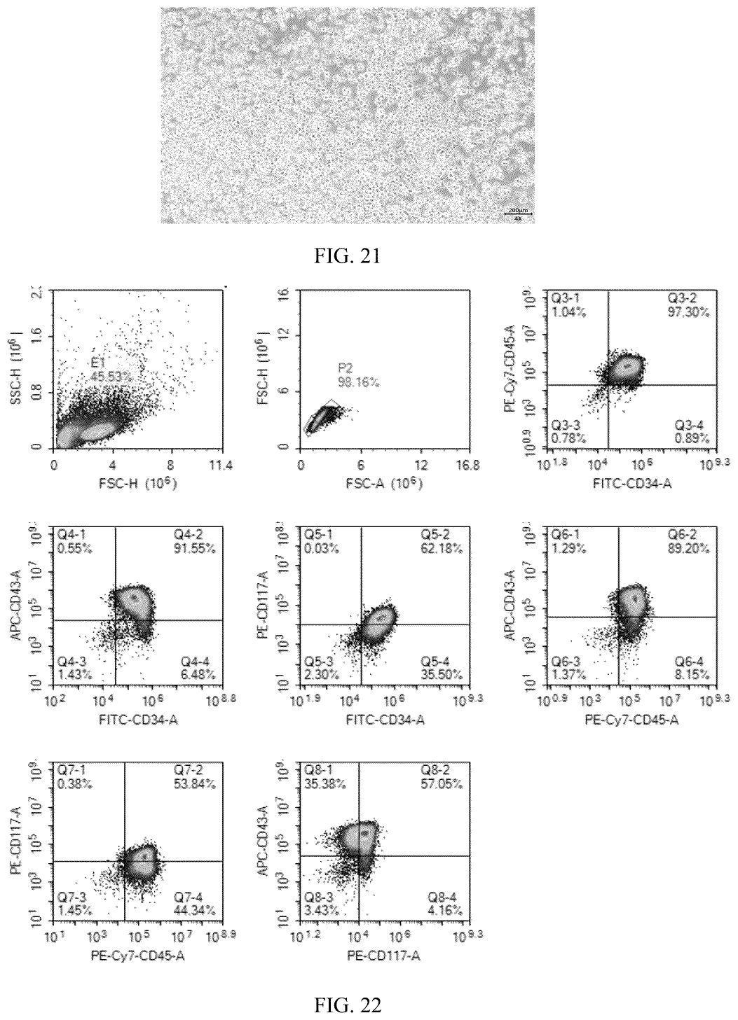

The hematopoietic progenitor cells obtained at the end of differentiation were observed under a microscope, and the morphology thereof is shown in FIG. 21 (4×). The hematopoietic progenitor cells obtained at the end of differentiation were subjected to flow cytometry analysis to determine the expression of markers. As shown in FIG. 22, the flow cytometric results of the hematopoietic progenitor cell stage after another 6 days of differentiation (on D12 of differentiation) are as follows: the proportion was 97.30% for CD34+/CD45+, 91.55% for CD34+/CD43+, 89.20% for CD45+/CD43+, 62.18% for CD34+/CD117+, 53.84% for CD45+/CD117+, and 57.05% for CD43+/CD117+.

### Example 4. Preparation of Hematopoietic Progenitor Cells

The procedures of this example are substantially the same as those of Example 1, except that the media of various stages are as follows:

### 1.1 D-1 Medium: E8 Medium+10 μM of Y27632

### 1.2 Stage I: Induced Mesoderm Differentiation (D0-D1)

Stage I medium: RPM1640 medium+2% of vitamin A-free B27 additive+1% of non-essential amino acids+1% glutamine+50 μg/mL of vitamin C+10 ng/mL of BMP4+5 μM of CHIR-99021.

### 1.3 Stage II: Induced Hemogenic Endothelium Differentiation (D2-D5)

Stage II medium: RPM1640 Medium+2% of vitamin A-free B27 additive+1% of non-essential amino acids+1% of glutamine+50 μg/mL of vitamin C+10 ng/mL of BMP4+20 ng/mL of VEGFA+20 ng/mL of FGF2+5 μM of SB431542.

### 1.4 Stage III: Induced Hematopoietic Progenitor Cell Differentiation (D6-D12)

Stage III medium: IMDM Medium+2% of vitamin A-free B27 additive+1% of NEAA+1% of GlutaMax+50 μg/mL of vitamin C+10 ng/mL of BMP4+10 ng/mL of VEGFA+50 ng/mL of SCF+30 μM of NAC (N-acetyl-L-cysteine)+2 μM of minocycline hydrochloride.

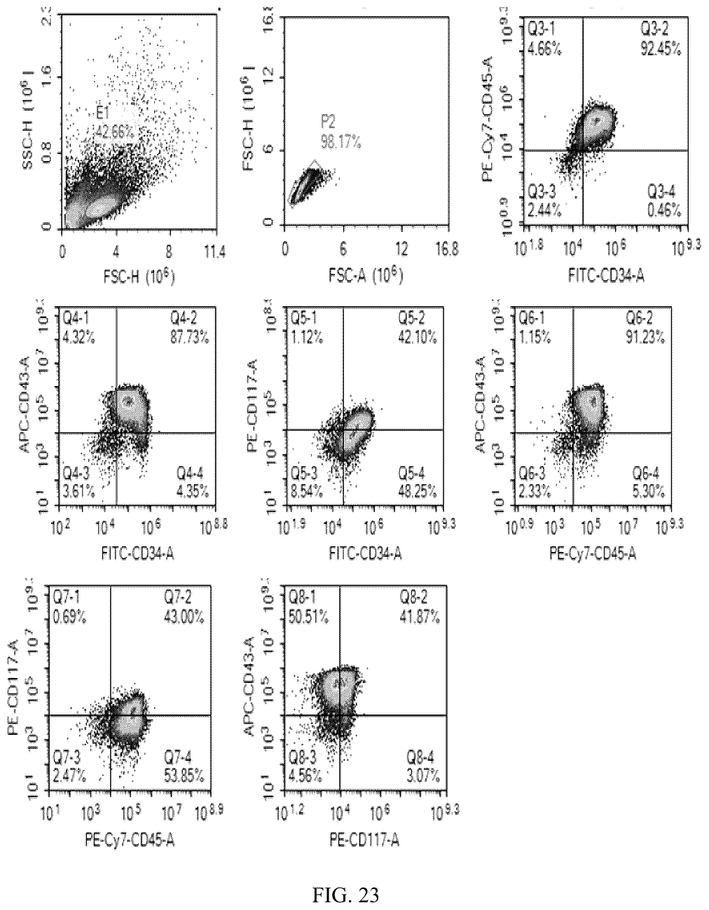

The hematopoietic progenitor cells obtained at the end of differentiation were subjected to flow cytometry analysis to determine the expression of markers. As shown in FIG. 23, the flow cytometric results of the hematopoietic progenitor cell stage after another 6 days of differentiation (on D12 of differentiation) are as follows: the proportion was 92.45% for CD34+/CD45+, 87.73% for CD34+/CD43+, 91.23% for CD45+/CD43+, 42.10% for CD34+/CD117+, 43% for CD45+/CD117+, and 41.87% for CD43+/CD117+.

### Example 5. Preparation of Hematopoietic Progenitor Cells

The procedures of this example are substantially the same as those of Example 1, except that the media and the culture time of various stages are as follows:

### 1.1 D-1 Medium: MTESR Medium+10 μM of Y27632

### 1.2 Stage I: Induced Mesoderm Differentiation (D0)

Stage I medium: RPM1640 medium+2% of vitamin A-free B27 additive+1% of non-essential amino acids+1% glutamine+50 μg/mL of vitamin C+5 μM of CHIR-99021.

### 1.3 Stage II: Induced Hemogenic Endothelium Differentiation (D1-D4)

Stage II medium: RPM1640 Medium+2% of vitamin A-free B27 additive+1% of non-essential amino acids+1% of glutamine+50 μg/mL of vitamin C+5 ng/mL of BMP4+50 ng/mL of VEGFA+50 ng/mL of FGF2+10 μM of SB431542.

### 1.4 Stage III: Induced Hematopoietic Progenitor Cell Differentiation (D5-D12)

Stage III medium: α-MEM medium+2% of vitamin A-free B27 additive+1% of NEAA+1% of GlutaMax+insulin-transferrin-selenium (ITS-G, 100×) 50 μg/mL of vitamin C+5 ng/mL of BMP4+10 ng/mL of VEGFA+50 ng/mL of SCF+30 μM of NAC (N-acetyl-L-cysteine)+2 μM of minocycline hydrochloride.

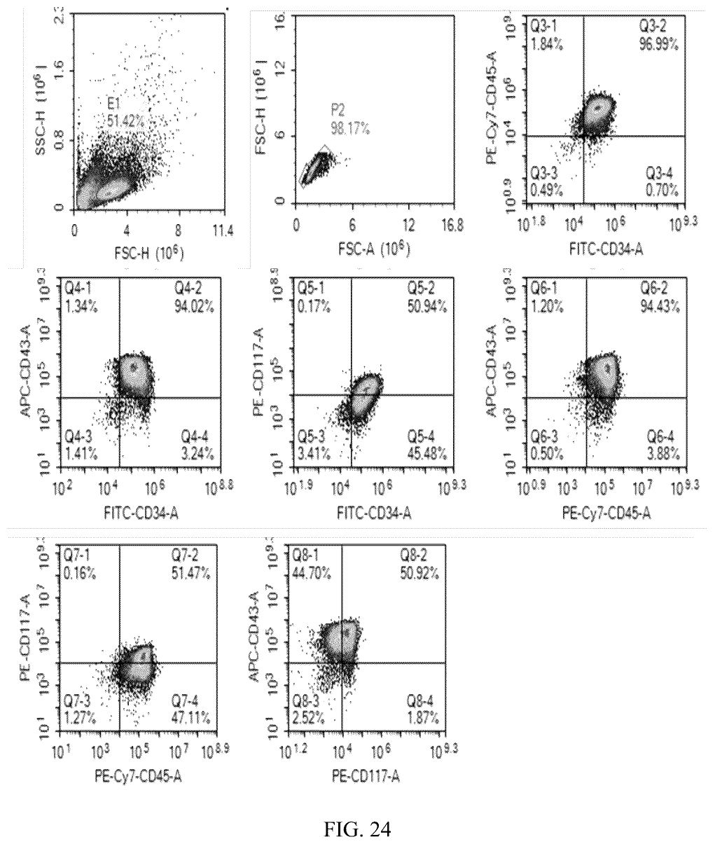

The hematopoietic progenitor cells obtained at the end of differentiation were subjected to flow cytometry analysis to determine the expression of markers. As shown in FIG. 24, the flow cytometric results of the hematopoietic progenitor cell stage after another 6 days of differentiation (on D12 of differentiation) are as follows: the proportion was 96.99% for CD34+/CD45+, 94.02% for CD34+/CD43+, 94.43% for CD45+/CD43+, 50.94% for CD34+/CD117+, 51.47% for CD45+/CD117+, and 50.92% for CD43+/CD117+.

### Example 6

1. The procedures for hematopoietic progenitor cell differentiation, flow cytometry analysis, and cell counting are the same as those in Example 1, and were repeated using different batches of H1 cells.

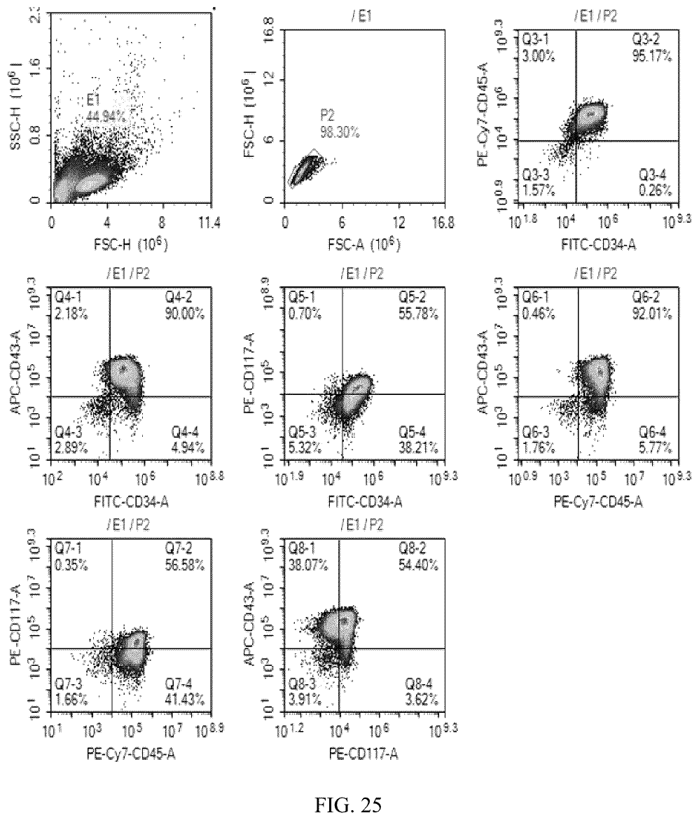

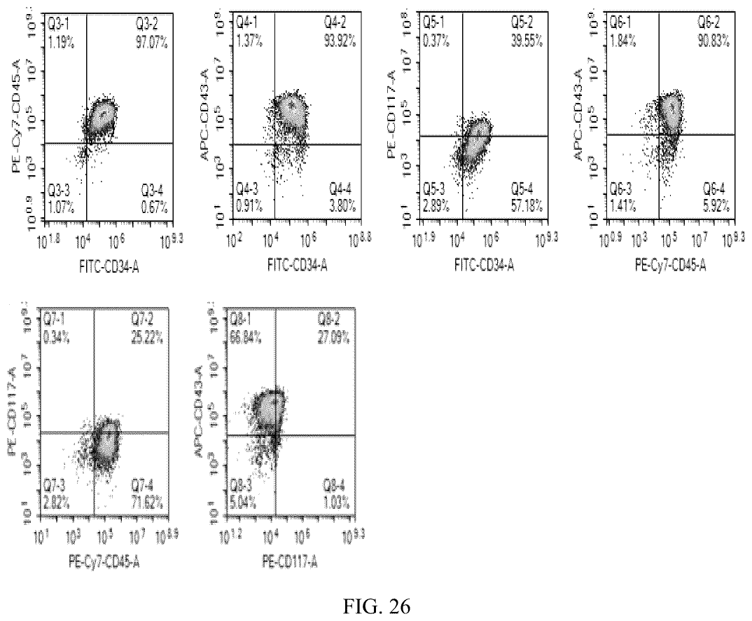

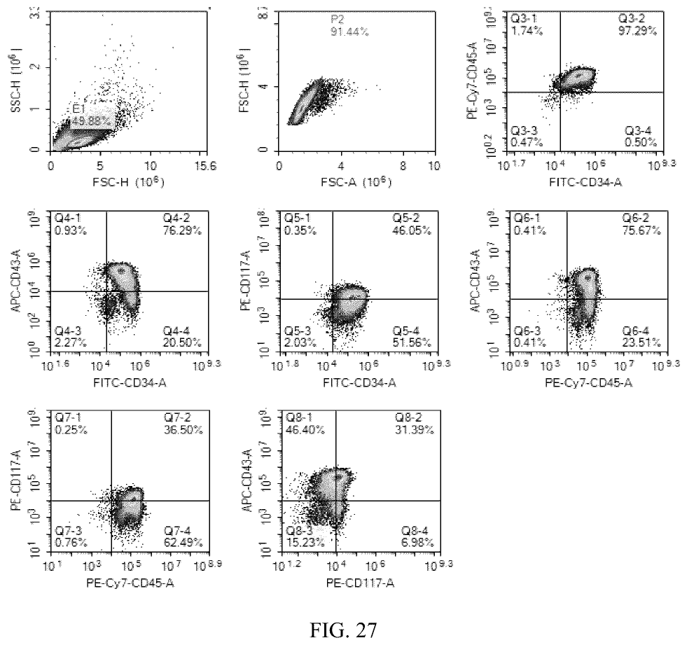

The final results are shown in Table 3. The flow cytometric graphs of hematopoietic progenitor cells obtained at the end of differentiation are shown in FIGS. 25-27. According to the results, the differentiation method of the present disclosure possesses good repeatability, stable differentiation effects, and a differentiation efficiency of 90% or greater, and can acquire a large quantity of hematopoietic progenitor cells.

TABLE 3
Results for hematopoietic progenitor cell differentiation

| | Initial | AO/PI | | Hematopoietic |
|---|---|---|---|---|
| | cell count | cell count | CD34+CD45+ | progenitor cell count |
| H1-P6 | 8*10\hat{ }4 | 2.48*10\hat{ }6 | 95.17% | 2.36*10\hat{ }6 |
| H1-P7 | 8*10\hat{ }4 | 3.28*10\hat{ }6 | 97.07% | 3.18*10\hat{ }6 |
| H1-P8 | 8*10\hat{ }4 | 3.15*10\hat{ }6 | 97.29% | 3.06*10\hat{ }6 |

### Comparative Example 1

The procedures for preparing hematopoietic progenitor cells are the same as those in Example 1, except that: monothioglycerol and penicillin-streptomycin were supplemented at 0.1 mM and 1% respectively in the Stage I, Stage II, and Stage III culture media.

The cell count and marker expression proportion of the hematopoietic progenitor cell obtained in Comparative Example 1 were determined according to the methods described in Example 1. The results show that the differentiation efficiency and the cell count of hematopoietic progenitor cells of Comparative Example 1 were significantly lower than those of the examples, suggesting that removal of monothioglycerol and penicillin-streptomycin from the media of various stages in the present disclosure can significantly improve the quantity of the hematopoietic progenitor cells and the differentiation efficiency.

The examples described above are preferred embodiments of the present disclosure, which, however, are not intended to limit the embodiments of the present disclosure. Any other changes, modifications, substitutions, combinations, and simplifications can be made without departing from the spirit and principle of the present disclosure, and should be construed as equivalent replacements and included in the protection scope of the present disclosure.

1. A method for preparing a hematopoietic progenitor cell, comprising:
(1) culturing a pluripotent stem cell to give an embryoid body;
(2) subjecting the embryoid body to mesoderm differentiation culture to give a mesodermal cell;
(3) subjecting the mesodermal cell to hemogenic endothelium differentiation culture to give a hemogenic endothelial cell; and
(4) subjecting the hemogenic endothelial cell to hematopoietic progenitor cell differentiation culture to give a hematopoietic progenitor cell,
wherein the method prepares hematopoietic progenitor cells in the absence of antibiotics and/or in the absence of monothioglycerol.

2. The method according to claim 1, wherein the culture system in step (1) comprises a ROCK inhibitor.

3. The method according to claim 1, wherein the culture system in step (2) comprises BMP4 and/or a GSK-3β inhibitor.

4. The method according to claim 1, wherein the culture system in step (3) comprises at least one selected from the group consisting of: BMP4, vascular endothelial growth factor, fibroblast growth factor, and a TGFβ/ALK inhibitor.

5. The method according to claim 1, wherein the culture system in step (4) comprises at least one selected from the group consisting of: BMP4, vascular endothelial growth factor, and stem cell factor.

6. A kit for preparing a hematopoietic progenitor cell, comprising at least one selected from the group consisting of: an embryoid body culture system, a mesoderm differentiation culture system, a hemogenic endothelium differentiation culture system, and a hematopoietic progenitor cell differentiation culture system, wherein the culture systems are free of antibiotics and/or monothioglycerol.

7.-10. (canceled)

11. A hematopoietic progenitor cell, wherein 90% or greater of the cells have the hematopoietic progenitor cell surface antigen CD34+, and the hematopoietic progenitor cell is prepared by the method according to claim 1.

12. A hematopoietic progenitor cell, wherein 90% or greater of the cells have the hematopoietic progenitor cell surface antigen CD43+, and the hematopoietic progenitor cell is prepared by the method according to claim 1.

13. A hematopoietic progenitor cell, wherein 90% or greater of the cells have the hematopoietic progenitor cell surface antigen CD45+, and the hematopoietic progenitor cell is prepared by the method according to claim 1.

14. A hematopoietic progenitor cell, wherein 90% or greater of the cells have the hematopoietic progenitor cell surface antigen combination CD34+/CD45+, and the hematopoietic progenitor cell is prepared by the method according to claim 1.

15. A hematopoietic progenitor cell, wherein 90% or greater of the cells have the hematopoietic progenitor cell surface antigen combination CD34+/CD43+, and the hematopoietic progenitor cell is prepared by the method according to claim 1.

16. A hematopoietic progenitor cell, wherein 90% or greater of the cells have the hematopoietic progenitor cell surface antigen combination CD43+/CD45+, and the hematopoietic progenitor cell is prepared by the method according to claim 1.

17. A hematopoietic progenitor cell, wherein 30% or greater of the cells have the hematopoietic progenitor cell surface antigen combination CD45+/CD117+, and the hematopoietic progenitor cell is prepared by the method according to claim 1.

18. A hematopoietic progenitor cell, wherein 40% or greater of the cells have the hematopoietic progenitor cell surface antigen combination CD43+/CD117+, and the hematopoietic progenitor cell is prepared by the method according to claim 1.

19. A hematopoietic progenitor cell, wherein 30% or greater of the cells have the hematopoietic progenitor cell surface antigen combination CD43+/CD117+, and the hematopoietic progenitor cell is prepared by the method according to claim 1.

20. A pharmaceutical composition, comprising a hematopoietic progenitor cell, and a pharmaceutically acceptable carrier, wherein the hematopoietic progenitor cell comprises at least one selected from the group consisting of:
(i) 90% or greater of the cells have the hematopoietic progenitor cell surface antigen CD34+;
(ii) 90% or greater of the cells have the hematopoietic progenitor cell surface antigen CD43+;
(iii) 90% or greater of the cells have the hematopoietic progenitor cell surface antigen CD45+;
(iv) 90% or greater of the cells have the hematopoietic progenitor cell surface antigen combination CD34+/CD45+;
(v) 90% or greater of the cells have the hematopoietic progenitor cell surface antigen combination CD34+/CD43+;
(vi) 90% or greater of the cells have the hematopoietic progenitor cell surface antigen combination CD43+/CD45+;
(vii) 30% or greater of the cells have the hematopoietic progenitor cell surface antigen combination CD45+/CD117+;
(vii) 40% or greater of the cells have the hematopoietic progenitor cell surface antigen combination CD34+/CD117+; and
(ix) 30% or greater of the cells have the hematopoietic progenitor cell surface antigen combination CD43+/CD117+,
and the hematopoietic progenitor cell is prepared by the method according to claim 1.

21. The pharmaceutical composition according to claim 20, wherein the pharmaceutical composition is a liquid formulation, a cell-based formulation, or an intravenous injection.

22. A method for treating or preventing a hematological disorder, comprising:
(i) 90% or greater of the cells have the hematopoietic progenitor cell surface antigen CD34+;
(ii) 90% or greater of the cells have the hematopoietic progenitor cell surface antigen CD43+;
(iii) 90% or greater of the cells have the hematopoietic progenitor cell surface antigen CD45+;
(iv) 90% or greater of the cells have the hematopoietic progenitor cell surface antigen combination CD34+/CD45+;
(v) 90% or greater of the cells have the hematopoietic progenitor cell surface antigen combination CD34+/CD43+;
(vi) 90% or greater of the cells have the hematopoietic progenitor cell surface antigen combination CD43+/CD45+;
(vii) 30% or greater of the cells have the hematopoietic progenitor cell surface antigen combination CD45+/CD117+;
(vii) 40% or greater of the cells have the hematopoietic progenitor cell surface antigen combination CD34+/CD117+; and
(ix) 30% or greater of the cells have the hematopoietic progenitor cell surface antigen combination CD43+/CD117+,
and the hematopoietic progenitor cell is prepared by the method according to claim 1.

23. The method according to claim 22, wherein the hematological disorder comprises anemia, thrombocytopenia, leukemia, lymphoma, severe aplastic anemia, multiple myeloma, or a combination thereof.

24. The method for treating or preventing a hematological disorder, comprising:
administrating the pharmaceutical composition according to claim 20 with effective dosage to a subject in need.


SEQUENCE LISTING (32 sequences)

SEQ ID NO: 1 - DNA, 23 bp, synthetic construct
  misc_feature 1..23: note = Synthesized
  source 1..23: mol_type = other DNA
attctcaacactccaaactgtgc

SEQ ID NO: 2 - DNA, 21 bp, synthetic construct
  misc_feature 1..21: note = Synthesized
  source 1..21: mol_type = other DNA
actttagcttcgggtcaatgc

SEQ ID NO: 3 - DNA, 20 bp, synthetic construct
  misc_feature 1..20: note = Synthesized
  source 1..20: mol_type = other DNA
cagccccacaagatcaaccc

SEQ ID NO: 4 - DNA, 19 bp, synthetic construct
  misc_feature 1..19: note = Synthesized
  source 1..19: mol_type = other DNA
caccggagactccctggat

SEQ ID NO: 5 - DNA, 22 bp, synthetic construct
  misc_feature 1..22: note = Synthesized
  source 1..22: mol_type = other DNA
ctgtccccaatagtgcttatgg

SEQ ID NO: 6 - DNA, 22 bp, synthetic construct
  misc_feature 1..22: note = Synthesized
  source 1..22: mol_type = other DNA
gaataggctgctgaattgaggg

SEQ ID NO: 7 - DNA, 21 bp, synthetic construct
  misc_feature 1..21: note = Synthesized
  source 1..21: mol_type = other DNA
gcaacccctactatgccaacc

SEQ ID NO: 8 - DNA, 19 bp, synthetic construct
  misc_feature 1..19: note = Synthesized
  source 1..19: mol_type = other DNA
cagtggcgtcttggagaag

SEQ ID NO: 9 - DNA, 19 bp, synthetic construct
  misc_feature 1..19: note = Synthesized
  source 1..19: mol_type = other DNA
ccgcgctcatttctcgtca

SEQ ID NO: 10 - DNA, 21 bp, synthetic construct
  misc_feature 1..21: note = Synthesized
  source 1..21: mol_type = other DNA
acggagggaatagtctggtcc

SEQ ID NO: 11 - DNA, 20 bp, synthetic construct
  misc_feature 1..20: note = Synthesized
  source 1..20: mol_type = other DNA
gcaggaagatgaaccgctct

SEQ ID NO: 12 - DNA, 19 bp, synthetic construct
  misc_feature 1..19: note = Synthesized
  source 1..19: mol_type = other DNA
ccaggcactggtttgggaa

SEQ ID NO: 13 - DNA, 19 bp, synthetic construct
  misc_feature 1..19: note = Synthesized
  source 1..19: mol_type = other DNA
ctcgcccatagacctgtgg

SEQ ID NO: 14 - DNA, 19 bp, synthetic construct
  misc_feature 1..19: note = Synthesized
  source 1..19: mol_type = other DNA
gttctgcgcgaaagagcac

SEQ ID NO: 15 - DNA, 21 bp, synthetic construct
  misc_feature 1..21: note = Synthesized
  source 1..21: mol_type = other DNA
aactcattttgcggtcgctat

SEQ ID NO: 16 - DNA, 20 bp, synthetic construct
  misc_feature 1..20: note = Synthesized
  source 1..20: mol_type = other DNA
tccctgaattgctcgctcac

SEQ ID NO: 17 - DNA, 19 bp, synthetic construct
  misc_feature 1..19: note = Synthesized
  source 1..19: mol_type = other DNA
tcccggacaagacgtacac

SEQ ID NO: 18 - DNA, 20 bp, synthetic construct
  misc_feature 1..20: note = Synthesized
  source 1..20: mol_type = other DNA
cgccactgaggtccttatca

SEQ ID NO: 19 - DNA, 20 bp, synthetic construct
  misc_feature 1..20: note = Synthesized
  source 1..20: mol_type = other DNA
tacgtggactcgttcctgct

SEQ ID NO: 20 - DNA, 19 bp, synthetic construct
  misc_feature 1..19: note = Synthesized
  source 1..19: mol_type = other DNA
cgtcgccttggactggaag

SEQ ID NO: 21 - DNA, 21 bp, synthetic construct
  misc_feature 1..21: note = Synthesized
  source 1..21: mol_type = other DNA
gaaagcgtcacttggggaaaa

SEQ ID NO: 22 - DNA, 23 bp, synthetic construct
  misc_feature 1..23: note = Synthesized
  source 1..23: mol_type = other DNA
tgttcgattcgggagataattgg

SEQ ID NO: 23 - DNA, 20 bp, synthetic construct
  misc_feature 1..20: note = Synthesized
  source 1..20: mol_type = other DNA
ctgcccatcgctttcaaggt

SEQ ID NO: 24 - DNA, 23 bp, synthetic construct
  misc_feature 1..23: note = Synthesized
  source 1..23: mol_type = other DNA
gccgagtagttttcatcattgcc

SEQ ID NO: 25 - DNA, 21 bp, synthetic construct
  misc_feature 1..21: note = Synthesized
  source 1..21: mol_type = other DNA
gacgtgagcatgtaccctagc

SEQ ID NO: 26 - DNA, 19 bp, synthetic construct
  misc_feature 1..19: note = Synthesized
  source 1..19: mol_type = other DNA
gcgtagccattccagtcct

SEQ ID NO: 27 - DNA, 19 bp, synthetic construct
  misc_feature 1..19: note = Synthesized
  source 1..19: mol_type = other DNA
gtggaccgcacggaatttg

SEQ ID NO: 28 - DNA, 20 bp, synthetic construct
  misc_feature 1..20: note = Synthesized
  source 1..20: mol_type = other DNA
ggagattcacaccggagtca

SEQ ID NO: 29 - DNA, 23 bp, synthetic construct
  misc_feature 1..23: note = Synthesized
  source 1..23: mol_type = other DNA
aatcctctcgtcaaaactgaagg

SEQ ID NO: 30 - DNA, 23 bp, synthetic construct
  misc_feature 1..23: note = Synthesized
  source 1..23: mol_type = other DNA
ccatctcgcttatccaacaatga

SEQ ID NO: 31 - DNA, 21 bp, synthetic construct
  misc_feature 1..21: note = Synthesized
  source 1..21: mol_type = other DNA
ggagcgagatccctccaaaat

SEQ ID NO: 32 - DNA, 23 bp, synthetic construct
  misc_feature 1..23: note = Synthesized
  source 1..23: mol_type = other DNA
ggctgttgtcatacttctcatgg